SEQUENCE LISTING (1 sequence)

SEQ ID NO: 1 - protein, 489 aa, Artificial Sequence
  Synthetic
MALPVTALLLPLALLLHAARPQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKSGAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGS

(19) United States
(12) Patent Application Publication
(10) Pub. No.: US 2023/0414755 A1
(43) Pub. Date: Dec. 28, 2023

(54) METHODS AND COMPOSITIONS RELATING TO GENETICALLY ENGINEERED CELLS EXPRESSING CHIMERIC ANTIGEN RECEPTORS

(71) Applicant: VOR BIOPHARMA INC., Cambridge, MA (US)

(72) Inventors: Sadik Kassim, Cambridge, MA (US); Julian Scherer, Cambridge, MA (US); Giacomo Canesin, Cambridge, MA (US); Tirtha Chakraborty, Cambridge, MA (US)

(73) Assignee: VOR BIOPHARMA INC., Cambridge, MA (US)

(21) Appl. No.: 18/036,698

(22) PCT Filed: Nov. 12, 2021
(86) PCT No.: PCT/US2021/059182

§ 371 Date: May 12, 2023

Related U.S. Application Data
(60) Provisional application No. 63/229,017, filed on Aug. 3, 2021.
(60) Provisional application No. 63/113,739, filed on Nov. 13, 2020.

(51) Int. Cl. A61K 39/00; C12N 9/22; C12N 15/11; C12N 15/90; A61P 35/02; C12N 5/0789; A61K 35/28

(52) U.S. Cl. CPC A61K 39/464411; C12N 9/22; C12N 15/111; C12N 15/907; A61P 35/02; C12N 5/0647; A61K 39/4611; A61K 35/28; A61K 39/4631; C12N 2310/20; C12N 2310/315; C12N 2310/321; A61K 2239/48; A61K 2239/38; A61K 2239/26

(57) ABSTRACT

The disclosure is directed to methods and compositions relating to genetically engineered cells expressing chimeric antigen receptors, where the cells are mobilized lymphocytes.

### RELATED APPLICATIONS

This application claims the benefit under 35 U.S.C. 119(e) of U.S. provisional application No. 63/113,739, filed Nov. 13, 2020, and U.S. provisional application No. 63/229,017, filed Aug. 3, 2021, which are incorporated by reference herein in their entireties.

### SUMMARY

The disclosure is directed, at least in part, to a genetically engineered cell comprising a heterologous nucleic acid encoding a chimeric antigen receptor (CAR) targeting a lineage-specific cell-surface antigen associated with a hyperproliferative disease, wherein the cell is a lymphocyte cell (or a descendant thereof) obtained from a subject after hematopoietic stem cell mobilization. In one aspect, the genetically engineered cell is a mobilized lymphocyte cell or a descendant of a mobilized lymphocyte cell. The disclosure is further directed, in part, to a method of producing such genetically engineered cells, and to methods of administering such genetically engineered cells (or a descendant thereof) to a subject in need thereof. The disclosure is based, in part, on the discovery that genetically engineered cells of the disclosure (e.g., CAR-expressing cells) that target a lineage-specific cell-surface antigen associated with a hyperproliferative disease can be generated from an apheresis sample from a subject who has undergone hematopoietic stem cell mobilization. In some embodiments, the methods described herein allow production of genetically engineered lymphocytes, e.g., CAR-expressing lymphocytes, and genetically engineered hematopoietic stem cells (HSCs) from the same apheresis sample taken from a subject after hematopoietic stem cell mobilization in the subject, thus avoiding multiple apheresis procedures, and creating HSCs, e.g., genetically engineered HSCs, that can be used for a hematopoietic stem cell transplant to a subject, as well as lymphocytes that are genetically matched to the HSCs, e.g., genetically engineered lymphocytes that express a CAR, and that can be administered to the same subject receiving the hematopoietic stem cell transplant.

Without wishing to be bound by any particular theory, hematopoietic stem cells are typically harvested from an apheresis sample taken from a subject after hematopoietic stem cell mobilization. However, only a subset of the cells of the apheresis sample (e.g., CD34+) cells are hematopoietic stem cells. Such apheresis samples also comprise peripheral blood mononuclear cells (PBMCs), which include, e.g., lymphocytes, and other cells. The disclosure is directed, in part, to the discovery that cells from the apheresis samples not harvested as hematopoietic stem cells (e.g., PBMCs) may be used to prepare a genetically engineered cell, e.g., a lymphocyte that expresses a chimeric antigen receptor (e.g., CAR cells). Such genetically engineered cells (e.g., CAR cells) can be used as immunotherapeutics and have uses in treating a number of diseases. In addition, the cells and methods of the disclosure can provide a resource efficient means to produce hematopoietic stem cells and genetically engineered cells of the disclosure (e.g., CAR cells) from a single apheresis sample from a single donor subject.

Aspects of the present disclosure provide genetically engineered cells comprising a heterologous nucleic acid encoding a chimeric antigen receptor (CAR) targeting lineage-specific cell-surface antigen associated with a hyperproliferative disease, wherein the cell is a lymphocyte cell, and wherein the cell, or a parental cell thereof, is obtained from a subject after hematopoietic stem cell mobilization. Aspects of the present disclosure related to genetically engineered cells comprising a heterologous nucleic acid encoding a chimeric antigen receptor (CAR) targeting lineage-specific cell-surface antigen associated with a hyperproliferative disease, wherein the cell is a mobilized lymphocyte cell, or a descendant thereof.

In some embodiments, the genetically engineered cells cell is a lymphocyte cell, and wherein the cell, or a parental cell thereof, is obtained from a subject after hematopoietic stem cell mobilization. In some embodiments, the cell is a mobilized lymphocyte cell.

In some embodiments, the genetically engineered cell or mobilized lymphocyte is a T lymphocyte. In some embodiments, the T lymphocyte expresses CD3, CD4, and/or CD8 (e.g., CD4 and CD8). In some embodiments, the T lymphocyte expresses PD1. In some embodiments, the T lymphocyte is an alpha/beta T lymphocyte, a gamma/delta T lymphocyte, a naïve T lymphocyte, an effector T lymphocyte, a memory T lymphocyte, or a regulatory T lymphocyte (Treg).

In some embodiments, the genetically engineered cell or mobilized lymphocyte is a B-lymphocyte. In some embodiments, the genetically engineered cell or mobilized lymphocyte is a natural killer (NK) cell.

In some embodiments, hematopoietic stem cell mobilization comprises administering to the subject etoposide, plerixafor, cyclophosphamide, and/or granulocyte colony-stimulating factor (G-CSF).

In some embodiments, the chimeric antigen receptor is a first generation CAR, second generation CAR, or a third generation CAR.

In some embodiments, the lineage-specific cell-surface antigen is CD33, CD30, CD38, CD123, CLL-1, CD5, CD6, CD7, CD19, or BCMA.

In some embodiments, the hyperproliferative disease is a hematopoietic malignancy or a myeloid malignancy. In some embodiments, the hematopoietic malignancy is Hodgkin's lymphoma, non-Hodgkin's lymphoma, leukemia, or multiple myeloma. In some embodiments, the leukemia is acute myeloid leukemia, acute lymphoid leukemia, myelodysplastic syndrome, chronic myelogenous leukemia, acute lymphoblastic leukemia or chronic lymphoblastic leukemia, or chronic lymphoid leukemia. In some embodiments, the hematopoietic malignancy is acute myeloid leukemia, myelodysplastic syndrome, or a lymphoid malignancy.

In some embodiments, the genetically engineered cell is for administration to a subject in need thereof, wherein the subject has, or has been diagnosed with a hematopoietic malignancy. In some embodiments, the subject has received a hematopoietic stem cell transplant comprising genetically engineered stem cells that have reduced expression or lack expression of the lineage-specific cell surface antigen or express a variant form of the lineage-specific cell-surface antigen that is not recognized or is recognized at a reduced level by the CAR.

In some aspects, the present disclosure provides methods comprising contacting a mobilized lymphocyte obtained from a first subject with a heterologous nucleic acid encoding a chimeric antigen receptor (CAR) targeting a lineage-specific cell-surface antigen associated with a hyperproliferative disease, thereby producing a genetically engineered lymphocyte expressing the CAR. In some embodiments, a method further comprises administering the genetically engineered lymphocyte, or a descendant thereof, to a second subject, wherein the second subject is in need thereof. In some embodiments, the second subject has, or has been diagnosed with, the hyperproliferative disease. In some embodiments, the first subject is different from the second subject. In some embodiments, the first subject is the same as the second subject.

In some embodiments, the subject in need of administering the genetically engineered lymphocyte, or a descendant thereof, has received a hematopoietic stem cell transplant comprising genetically engineered hematopoietic stem cells that have reduced expression or lack expression of the lineage-specific cell surface antigen, or express a variant form of the lineage-specific cell-surface antigen that is not recognized or is recognized at a reduced level by the CAR. In some embodiments, a method further comprises administering the hematopoietic stem cell transplant to the subject in need thereof after administration of the genetically engineered lymphocyte, or a descendant thereof. In some embodiments, the genetically engineered lymphocyte, or a descendant thereof, is administered in combination with the hematopoietic stem cell transplant comprising genetically engineered hematopoietic stem cells.

In some embodiments, the method further comprises contacting a hematopoietic stem cell obtained from the first subject with an RNA-guided nuclease and guide RNA or a nucleic acid encoding the same, wherein the guide RNA targets a gene encoding the lineage-specific cell-surface antigen, thereby producing a genetically engineered hematopoietic stem cell that has reduced expression or lacks expression of the lineage-specific cell-surface antigen or expresses a variant form of the lineage-specific cell-surface antigen. In some embodiments, the genetically engineered hematopoietic stem cell is not targeted by a CAR, e.g., the CAR of a genetically engineered lymphocyte. In some embodiments, the method further comprises administering the genetically engineered hematopoietic stem cell, or a descendant thereof, to a subject in need thereof.

In some embodiments, the genetically engineered cell or mobilized lymphocyte is a T lymphocyte. In some embodiments, the T lymphocyte expresses CD3, CD4, and/or CD8 (e.g., CD4 and CD8). In some embodiments, the T lymphocyte expresses PD1. In some embodiments, the T lymphocyte is an alpha/beta T lymphocyte, a gamma/delta T lymphocyte, a naïve T lymphocyte, an effector T lymphocyte, a memory T lymphocyte, or a regulatory T lymphocyte (Treg).

In some embodiments, the genetically engineered cell or mobilized lymphocyte is a B-lymphocyte. In some embodiments, the genetically engineered cell or mobilized lymphocyte is a natural killer (NK) cell.

In some embodiments, the lineage-specific cell-surface antigen is CD33, CD30, CD38, CD123, CLL-1, CD5, CD6, CD7, CD19, or BCMA. In some embodiments, the chimeric antigen receptor is a first generation CAR, second generation CAR, or a third generation CAR.

In some embodiments, the hyperproliferative disease is a hematopoietic malignancy or a myeloid malignancy. In some embodiments, the hematopoietic malignancy is Hodgkin's lymphoma, non-Hodgkin's lymphoma, leukemia, or multiple myeloma. In some embodiments, the leukemia is acute myeloid leukemia, acute lymphoid leukemia, myelodysplastic syndrome, chronic myelogenous leukemia, acute lymphoblastic leukemia or chronic lymphoblastic leukemia, or chronic lymphoid leukemia. In some embodiments, the hematopoietic malignancy is acute myeloid leukemia, myelodysplastic syndrome, or a lymphoid malignancy.

In some embodiments, the RNA-guided nuclease is a CRISPR/Cas nuclease. In some embodiments, the CRISPR/Cas nuclease is a Cas9 nuclease, a SpCas nuclease, a SaCas nuclease, or a Cpf1nuclease. In some embodiments, the nucleic acid encoding the guide RNA and/or the RNA-guided nuclease is an RNA, preferably an mRNA or an mRNA analog. In some embodiments, the guide RNA comprises one or more nucleotide residues that are chemically modified. In some embodiments, the guide RNA comprises one or more nucleotide residues that comprise a 2′O-methyl moiety. In some embodiments, the guide RNA comprises one or more nucleotide residues that comprise a phosphorothioate. In some embodiments, the guide RNA comprises one or more nucleotide residues that comprise a thioPACE moiety.

In some aspects, the present disclosure provides cell populations comprising a plurality of the genetically engineered cells disclosed herein, or produced, obtained, or obtainable by a method described herein. In some aspects, the present disclosure provides methods comprising administering any of the cell populations described herein to a subject in need thereof.

### BRIEF DESCRIPTION OF THE DRAWINGS

The accompanying drawings, which constitute a part of this specification, illustrate several embodiments of the invention and together with the description, serve to explain the principles of the invention.

FIG. 1 is a schematic showing an exemplary embodiment of production of edited hematopoietic stem cells (eHSCs) and chimeric antigen receptor (CAR)-expressing cell treatment system from a single donor.

FIG. 2 is a schematic showing an exemplary method in which edited hematopoietic stem cells (eHSCs) and chimeric antigen receptor (CAR)-expressing T cells (CAR-T cells) are produced from a single donor and administered to a patient.

FIGS. 3A-3H show plots characterizing the population of mobilized peripheral blood mononuclear cells (PBMCs, referred to as the “non-target fraction” in FIG. 2). FIG. 3A is a flow cytometric analysis plot showing the population of CD3+ cells (T cells). FIG. 3B is a flow cytometric analysis plot showing the population of CD14+ cells (monocytes). FIG. 3C is a flow cytometric analysis plot showing the population of CD56+ cells (NK cells). FIG. 3D is a flow cytometric analysis plot showing the population of CD19+ cells (B cells). FIG. 3E presents charts showing the relative abundance of CD3+ cells (T cells), monocytes, NK cells, and B cells in a steady state cell fraction (left panel) or a mobilized cell fraction (right panel). FIG. 3F is a flow cytometric analysis plot showing further characterization of the population of CD3+ cells (T cells) of FIG. 3A based on expression of CCR7 (y-axis) and CD45RA (x-axis). FIG. 3G is a schematic showing the identification of cell types of FIG. 3F. TCM: central memory T cells, low CD45RA, high CCR7; Naïve: high CD45RA, high CCR7; TEM: effector memory T cells, low CD45RA, low CCR7; TEFF: effector T cells, high CD45RA, low CCR7. FIG. 3H presents charts showing the relative abundance of T cell populations a steady state cell fraction (left panel) or a mobilized cell fraction (right panel).

FIGS. 4A-4D show immunophenotypic characterization of mobilized and non-mobilized PBMC populations. FIG. 4A: immune cell phenotyping showing CD3+ cells, monocytes, NK cells, NKT cells, and B cells. FIG. 4B: T cell phenotype comparison showing CD8 and CD4 populations. FIG. 4C: T cell phenotype comparison showing naïve T cells, T effector cells (Teff), T effector memory cells (TEM), and central memory T cell (TCM) populations. FIG. 4D: steady state and mobilized CD8+ and CD4+ subsets. For each of cell type of FIGS. 4A-4C, the data points correspond to steady state (left) and mobilized (right).

FIG. 5 shows CITEseq characterization of PBMC populations of two donors (D1 and D2). In the left panel stacked graphs, each column from top to bottom corresponds to B cell, CD4+ T cell, CD8+ T cells, HSPCs, macrophage, monocytes, nature killer cell, and pDC populations. In the right panel, for each cell type, the left column refers to mobilized cells, and the right column refers to steady state cells.

FIG. 6 shows CITEseq characterization of T-cell sub-populations of two donors (D1 and D2). In the left panel stacked graphs, each column from top to bottom corresponds to CD4+ T effector cell, CD4+ T naïve, CD4+ TCM, CD4+ TEM, CD8+ T effector cells, and CD8+ T naïve cell populations. In the right panel, for each cell type, the left column refer to mobilized cells, and the right column corresponds to stead state cells.

FIG. 7 shows characterization of T-cell activation in lymphocytes obtained from mobilized and non-mobilized PBMC populations from two donors (D1 and D2). For each marker, the columns refer, from left to right, to steady state(SS) unactivated, mobilized (Mob) unactivated, SS activated, and Mob activated.

FIG. 8 shows characterization of T-cell activation in lymphocytes obtained from mobilized and non-mobilized PBMC populations across a larger donor cohort.

FIG. 9 shows results of a cytotoxicity assay using mobilized (mob) and non-mobilized (ss) PBMC-derived cells transduced with an anti-CD33 CAR. For each agent, each column corresponds, from top to bottom, to dead, apoptotic, and live cells.

FIG. 10 shows intracellular cytokine staining (ICS) analysis of TNFalpha (TNFa+) and IFNgamma (IFNg+) expression in mobilized and non-mobilized PBMC-derived anti-CD33 CAR expressing cell populations in the presence and absence of CD33-expressing target cells. For each condition, the columns correspond, from left to right, to no stimulation (No Stim), PMA/I, WT MOLM13, CD33KO MOLM13, and Jurkat cells.

FIG. 11 shows results from LUMINEX™ analysis of TNFalpha (TNFa) and IFNgamma (IFNg) expression in mobilized and non-mobilized PBMC-derived anti-CD33 CAR expressing cell populations in the presence and absence of CD33-expressing target cells. For each condition, the columns correspond, from left to right, to no stimulation (No Stim), PMA/I, WT MOLM13, CD33KO MOLM13, and Jurkat cells.

FIG. 12 shows results of a cytotoxicity assay of mobilized PBMC-derived anti-CD33 CAR T cells and non-mobilized PBMC-derived anti-CD33 CAR T cells in an in vivo mouse model.

### DETAILED DESCRIPTION

Hematopoietic stem cells (HSCs) for clinical use can be obtained from donor subjects after hematopoietic stem cell mobilization via apheresis. The apheresis sample obtained from such subjects typically includes a fraction of HSCs, e.g., CD34+ HSCs, sometimes also referred to as the “target fraction,” and a fraction of cells that are not stem cells, e.g., peripheral blood mononuclear cells (PBMCs) and other cells, also sometimes referred to as the “non-target fraction.” Some therapeutic approaches utilize genetically engineered HSCs, e.g., edited HSCs that lack expression of a lineage-specific antigen, in combination with immunotherapeutics, such as lymphocytes expressing a chimeric antigen receptor (CAR) targeting the lineage-specific antigen, in order to specifically eradicate cells that are associated with malignant disease, while sparing non-malignant hematopoietic cells. See, e.g., Borot et al., Gene-edited stem cells enable CD33-directed immune therapy for myeloid malignancies PNAS 2019, 116(24):11978-11987; and international patent application PCT/US16/057339, published as WO 2017/066760. In such approaches, a subject is typically administered a hematopoietic cell transplant (HCT) comprising the genetically engineered HSCs, and after successful engraftment of the HSCs in the subject, the immunotherapeutic, e.g., CAR-T cells, is administered to the subject.

The present disclosure is directed, in part, to the discovery that mobilized lymphocytes may be used to produce genetically engineered cells (e.g., CAR cells). In some embodiments, compositions and methods are provided that relate to producing genetically engineered hematopoietic stem cells and genetically engineered lymphocytes, e.g., CAR cells, from the same subject, e.g., from the same blood (e.g., apheresis) sample from the subject, thus efficiently providing two different cell populations for treatment from a single sample from the donor subject. The disclosure is directed in part to genetically engineered cells comprising a heterologous nucleic acid encoding a CAR targeting lineage-specific cell-surface antigen associated with a hyperproliferative disease, wherein the cell is a mobilized lymphocyte (or a descendant of a mobilized lymphocyte) or is a lymphocyte (or a descendant thereof) obtained from a subject after hematopoietic stem cell mobilization. Also included herein are methods for producing and administering said cells (e.g., a genetically engineered HSC, e.g., lacking expression of the lineage-specific antigen, and a genetically engineered lymphocyte, e.g., expressing a CAR targeting the lineage-specific antigen, obtained from a single apheresis sample from a subject after hematopoietic stem cell mobilization) to a subject in need thereof, e.g., a subject having a cancer characterized by expression of the lineage-specific antigen, wherein the lineage-specific antigen is also expressed on some non-malignant cells. The disclosure also provides methods of producing and/or administering genetically engineered hematopoietic stem cells, wherein the genetically engineered hematopoietic stem cells are not targeted by the CAR.

### Definitions

Antibody: As used herein, the term “antibody” refers to a polypeptide that includes canonical immunoglobulin sequence elements sufficient to confer specific binding to a particular target antigen. As is known in the art, intact antibodies as produced in nature are typically approximately 150 kD tetrameric agents comprising two identical heavy chain polypeptides (about 50 kD each) and two identical light chain polypeptides (about 25 kD each) that associate with each other into what is commonly referred to as a “Y-shaped” structure. Each heavy chain comprises at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain comprises two domains—an amino-terminal variable (VL) domain, followed by a carboxy-terminal constant (CL) domain, separated from one another by another “switch”. Intact antibody tetramers comprise two heavy chain-light chain dimers in which the heavy and light chains are linked to one another by a single disulfide bond; two other disulfide bonds connect the heavy chain hinge regions to one another, so that the dimers are connected to one another and a tetramer is formed. Naturally-produced antibodies are also typically glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complementarity determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including, for example, effector cells that mediate cytotoxicity. Affinity and/or other binding attributes of Fc regions for Fc receptors can be modulated through glycosylation or other modification. In some embodiments, antibodies produced and/or utilized in accordance with the present invention (e.g., as a component of a CAR) include glycosylated Fc domains, including Fc domains with modified or engineered glycosylation. In some embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal. In some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc., as is known in the art. Moreover, the term “antibody”, as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present invention is in a format selected from, but not limited to, intact IgA, IgG, IgE or IgM antibodies; bi- or multi-specific antibodies (e.g., Zybodies®, etc); antibody fragments such as is used herein in the broadest sense and encompasses various antibody structures, including but not limited to monoclonal antibodies, polyclonal antibodies, multispecific antibodies (e.g., bispecific antibodies), and/or antibody fragments (preferably those fragments that exhibit the desired antigen-binding activity). An antibody described herein can be an immunoglobulin, heavy chain antibody, light chain antibody, LRR-based antibody, or other protein scaffold with antibody-like properties, as well as other immunological binding moiety known in the art, including, e.g., a Fab, Fab′, Fab′2, Fab2, Fab3, F(ab′)2, Fd, Fv, Feb, scFv, SMIP, single domain antibody, single-chain antibody, diabody, tribody, tetrabody, minibody, maxibody, tandab, DVD, BiTe, TandAb, or the like, or any combination thereof. The subunit structures and three-dimensional configurations of different classes of antibodies are known in the art. In some embodiments, an antibody may lack a covalent modification (e.g., attachment of a glycan) that it would have if produced naturally. In some embodiments, an antibody may contain a covalent modification (e.g., attachment of a glycan, a payload (e.g., a detectable moiety, a therapeutic moiety, a catalytic moiety, etc.), or other pendant group (e.g., poly-ethylene glycol, etc.).

Antigen-binding fragment: An “antigen-binding fragment” refers to a portion of an antibody that binds the antigen to which the antibody binds. An antigen-binding fragment of an antibody includes any naturally occurring, enzymatically obtainable, synthetic, or genetically engineered polypeptide or glycoprotein that specifically binds an antigen to form a complex. Exemplary antibody fragments include, but are not limited to, Fv, Fab, Fab′, Fab′-SH, F(ab′)2; diabodies; single domain antibodies; linear antibodies; single-chain antibody molecules (e.g. scFv or VHH or VH or VL domains only); and multispecific antibodies formed from antibody fragments. In some embodiments, the antigen-binding fragments of the antibodies described herein are scFvs. In some embodiments, the antigen-binding fragments of the antibodies described herein are VHH domains only. As with full antibody molecules, antigen-binding fragments may be mono-specific or multispecific (e.g., bispecific). A multispecific antigen-binding fragment of an antibody may comprise at least two different variable domains, wherein each variable domain is capable of specifically binding to a separate antigen or to a different epitope of the same antigen.

Antibody heavy chain: As used herein, the term “antibody heavy chain” refers to the larger of the two types of polypeptide chains present in all antibody molecules in their naturally occurring conformations.

Antibody light chain: As used herein, the term “antibody light chain” refers to the smaller of the two types of polypeptide chains present in all antibody molecules in their naturally occurring conformations.

Synthetic antibody: As used herein, the term “synthetic antibody” refers to an antibody that is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

Antigen: As used herein, the term “antigen” or “Ag” refers to a molecule that is capable of provoking an immune response. This immune response may involve either antibody production, the activation of specific immunologically-competent cells, or both. A skilled artisan will understand that any macromolecule, including virtually all proteins or peptides, can serve as an antigen. Furthermore, antigens can be derived from recombinant or genomic DNA. A skilled artisan will understand that any DNA that comprises a nucleotide sequences or a partial nucleotide sequence encoding a protein that elicits an immune response encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

Autologous: As used herein, the term “autologous” refers to any material derived from an individual to which it is later to be re-introduced into the same individual.

Allogeneic: As used herein, the term “allogeneic” refers to any material (e.g., a population of cells) derived from a different animal of the same species.

Hyperproliferative disease: As used herein, the term “hyperproliferative disease” refers to a disease characterized by the rapid and uncontrolled growth of aberrant cells. A hyperproliferative disease may be a benign or a malign disease. Malign diseases are typically characterized by the presence of malign cells, e.g., cancer cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. In certain embodiments, the hyperproliferative is a hematopoietic malignancy, such as a myeloid malignancy or a lymphoid malignancy. In some embodiments, the hematopoietic malignancy is acute myeloid leukemia. In some embodiments, the hematopoietic malignancy is myelodysplastic syndrome.

Conservative sequence modifications: As used herein, the term “conservative sequence modifications” refers to amino acid modifications that do not significantly affect or alter the binding characteristics of an antibody containing the amino acid sequence. Such conservative modifications include amino acid substitutions, additions and deletions. Modifications can be introduced into an antibody compatible with various embodiments by standard techniques known in the art, such as site-directed mutagenesis and PCR-mediated mutagenesis. Conservative amino acid substitutions are ones in which an amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for the ability to bind antigens using the functional assays described herein.

Co-stimulatory ligand: As used herein, the term “co-stimulatory ligand” refers to a molecule on an antigen presenting cell (e.g., an APC, dendritic cell, B cell, and the like) that specifically binds a cognate co-stimulatory molecule on an immune cell (e.g., a T lymphocyte), thereby providing a signal which mediates an immune cell response, including, but not limited to, proliferation, activation, differentiation, and the like. A co-stimulatory ligand can include, but is not limited to, CD7, B7-1 (CD80), B7-2 (CD86), CD28, PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on an immune cell (e.g., a T lymphocyte), such as, but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

Cytotoxic: As used herein, the term “cytotoxic” or “cytotoxicity” refers to killing or damaging cells. In one embodiment, cytotoxicity of the metabolically enhanced cells is improved, e.g. increased cytolytic activity of immune cells (e.g., T lymphocytes).

Effective amount: As used herein, an “effective amount” as described herein refers to a dose that is adequate to prevent or treat a neoplastic disease, e.g., a cancer, in an individual. Amounts effective for a therapeutic or prophylactic use will depend on, for example, the stage and severity of the disease or disorder being treated, the age, weight, and general state of health of the patient, and the judgment of the prescribing physician. The size of the dose will also be determined by the active selected, method of administration, timing and frequency of administration, the existence, nature, and extent of any adverse side-effects that might accompany the administration of a particular active, and the desired physiological effect. It will be appreciated by one of skill in the art that various diseases or disorders could require prolonged treatment involving multiple administrations, perhaps using the genetically engineered cells of the disclosure (e.g., CAR cells) in each or various rounds of administration, for example in temporal proximity with edited hematopoietic stem cells, as described herein.

For purposes of the invention, the amount or dose of a genetically engineered cell comprising a heterologous nucleic acid comprising a CAR construct described herein that is administered should be sufficient to effect a therapeutic or prophylactic response in the subject or animal over a reasonable time frame. For example, the dose should be sufficient to bind to antigen, or detect, treat, or prevent cancer in a period of from about 2 hours or longer, e.g., about 12 to about 24 or more hours, from the time of administration. In certain embodiments, the time period could be even longer. The dose will be determined by the efficacy of the particular genetically engineered cells of the disclosure (e.g., CAR cells) and the condition of the animal (e.g., human), as well as the body weight of the animal (e.g., human) to be treated.

Effector function: As used herein, “effector function” or “effector activity” refers to a specific activity carried out by an immune cell in response to stimulation of the immune cell. For example, an effector function of a T lymphocyte includes, recognizing an antigen and killing a cell that expresses the antigen.

Endogenous: As used herein “endogenous” refers to any material from or produced inside a particular organism, cell, tissue or system.

Exogenous: As used herein, the term “exogenous” refers to any material introduced from or produced outside a particular organism, cell, tissue or system.

Expand: As used herein, the term “expand” refers to increasing in number, as in an increase in the number of cells, for example, immune cells, e.g., T lymphocytes, B lymphocytes, NK cells, and/or hematopoietic cells. In one embodiment, immune cells, e.g., T lymphocytes, B lymphocytes, NK cells, and/or hematopoietic cells that are expanded ex vivo increase in number relative to the number originally present in a culture. In another embodiment, immune cells, e.g., T lymphocytes, B lymphocytes, NK cells, and/or hematopoietic cells that are expanded ex vivo increase in number relative to other cell types in a culture. In some embodiments, expansion may occur in vivo. The term “ex vivo,” as used herein, refers to cells that have been removed from a living organism, (e.g., a human) and propagated outside the organism (e.g., in a culture dish, test tube, or bioreactor).

Functional Portion: As used herein, the term “functional portion” when used in reference to a CAR refers to any part or fragment of the CAR constructs of the invention, which part or fragment retains the biological activity of the CAR construct of which it is a part (the parent CAR construct). Functional portions encompass, for example, those parts of a CAR construct that retain the ability to recognize target cells, or detect, treat, or prevent cancer, to a similar extent, the same extent, or to a higher extent, as the parent CAR construct. In reference to the parent CAR construct, the functional portion can comprise, for instance, about 10%, about 25%, about 30%, about 50%, about 68%, about 80%, about 90%, about 95%, or more, of the parent CAR.

The functional portion can comprise additional amino acids at the amino or carboxy terminus of the portion, or at both termini, which additional amino acids are not found in the amino acid sequence of the parent CAR construct. Desirably, the additional amino acids do not interfere with the biological function of the functional portion, e.g., recognize target cells, detect cancer, treat or prevent cancer, etc. More desirably, the additional amino acids enhance the biological activity as compared to the biological activity of the parent CAR construct.

Functional Variant: As used herein, the term “functional variant,” as used herein, refers to a CAR construct, polypeptide, or protein having substantial or significant sequence identity or similarity to a parent CAR construct, which functional variant retains the biological activity of the CAR of which it is a variant. Functional variants encompass, for example, those variants of the CAR construct described herein (the parent CAR construct) that retain the ability to recognize target cells to a similar extent, the same extent, or to a higher extent, as the parent CAR construct. In reference to the parent CAR construct, the functional variant can, for instance, be at least about 30%, about 50%, about 75%, about 80%, about 90%, about 91%, about 92%, about 93%, about 94%, about 95%, about 96%, about 97%, about 98%, about 99% or more identical in amino acid sequence to the parent CAR construct. A functional variant can, for example, comprise the amino acid sequence of the parent CAR with at least one conservative amino acid substitution. Alternatively or additionally, the functional variants can comprise the amino acid sequence of the parent CAR construct with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent CAR construct.

gRNA: The terms “gRNA” and “guide RNA” are used interchangeably throughout and refer to a nucleic acid that promotes the specific targeting or homing of a gRNA/Cas9 molecule complex to a target nucleic acid. A gRNA can be unimolecular (having a single RNA molecule), sometimes referred to herein as sgRNAs, or modular (comprising more than one, and typically two, separate RNA molecules). A gRNA may bind to a target domain in the genome of a host cell. The gRNA (e.g., the targeting domain thereof) may be partially or completely complementary to the target domain. The gRNA may also comprise a “scaffold sequence,” (e.g., a tracrRNA sequence), that recruits a Cas9 molecule to a target domain bound to a gRNA sequence (e.g., by the targeting domain of the gRNA sequence). The scaffold sequence may comprise at least one stem loop structure and recruits an endonuclease. Exemplary scaffold sequences can be found, for example, in Jinek, et al. I (2012) 337(6096):816-821, Ran, et al. Nature Protocols (2013) 8:2281-2308, PCT Publication No. WO2014/093694, and PCT Publication No. WO2013/176772.

Heterologous: As used herein, the term “heterologous” refers to a phenomenon occurring in a living system, e.g., a cell, that does not naturally occur in that system. For example, expression of a protein in a cell, where the protein does not naturally occur in that cell (e.g., the cell does not naturally encode that protein), would be heterologous expression of the protein. In some embodiments, the heterologous nucleic acid encodes a chimeric antigen receptor construct.

Immune cell: As used herein, the term “immune cell,” used interchangeably herein with the term “immune effector cell,” refers to a cell that is involved in an immune response, e.g., promotion of an immune response. Examples of immune cells include, but are not limited to, T-lymphocytes, natural killer (NK) cells, macrophages, monocytes, dendritic cells, neutrophils, eosinophils, mast cells, platelets, large granular lymphocytes, Langerhans' cells, or B-lymphocytes. A source of immune cells (e.g., T lymphocytes, B lymphocytes, NK cells) can be obtained from a subject.

Immune response: As used herein the term “immune response” refers to a cellular and/or systemic response to an antigen that occurs when lymphocytes identify antigenic molecules as foreign and induce the formation of antibodies and/or activate lymphocytes to remove the antigen.

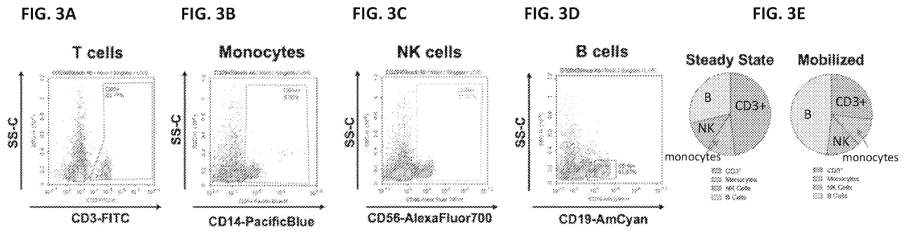

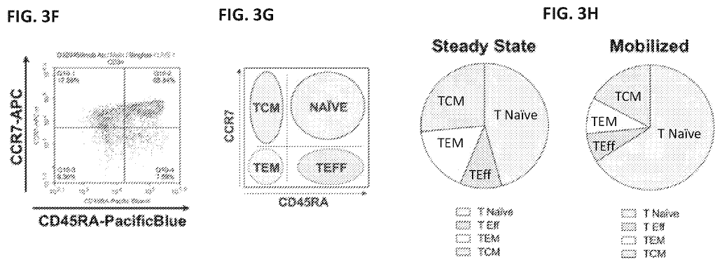

Mobilized cell/lymphocyte: As used herein, the term “mobilized cells” refers to cells obtained from a blood sample (e.g., an apheresis sample) from a subject that has undergone hematopoietic stem cell mobilization. In contrast, “steady state cells” refer to cells obtained from a blood sample (e.g., an apheresis sample) from a subject that has not undergone hematopoietic stem cell mobilization. As shown, for example, in FIGS. 3E and 3H, the relative populations of cell types and developmental state of the cells are different between steady state and mobilized samples. In some embodiments, a mobilized cells population comprises a higher proportion of B cells (B-lymphocytes) as compared to a steady state cell population. In some embodiments, a mobilized cell population comprises a lower proportion of T cells (T-lymphocytes) than steady state cells. In some embodiments, mobilized cells comprise a higher proportion of naïve T cells than steady state cells. In some embodiments, mobilized cells comprise a lower proportion of one, two, or all of central memory T cells (TCM), effector memory T cells (TEM), or T-cell effectors than steady state cells. As used herein, the term “mobilized lymphocyte” refers to a lymphocyte (or a descendant thereof) obtained from a subject that has undergone hematopoietic stem cell mobilization. Hematopoietic stem cell mobilization techniques and protocols are used routinely in the clinic, and exemplary techniques and protocols suitable in accordance with aspects of this disclosure include protocols that utilize etoposide, plerixafor, cyclophosphamide, and/or granulocyte colony-stimulating factor (G-CSF). Some exemplary protocols are provided herein, and additional suitable protocols will be apparent to those of skill in the art. Some exemplary suitable protocols include those recited in Albakri et al., A Review of Advances in Hematopoietic Stem Cell Mobilization and the Potential Role of Notch2 Blockade, Cell Transplant 2020, 29:0963689720947146.

Nucleic acid: As used herein, the term “nucleic acid” refers to a polymer of at least three nucleotides. In some embodiments, a nucleic acid comprises DNA. In some embodiments, a nucleic acid comprises RNA. In some embodiments, a nucleic acid is single stranded. In some embodiments, a nucleic acid is double stranded. In some embodiments, a nucleic acid comprises both single and double stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid”. In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises one or more, or all, non-natural residues. In some embodiments, a non-natural residue comprises a nucleoside analog (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a non-natural residue comprises one or more modified sugars (e.g., 2′-fluororibose, ribose, 2′-deoxyribose, arabinose, and hexose) as compared to those in natural residues. In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro, reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long.

Single chain antibodies: As used herein, the term “single chain antibodies” refers to antibodies formed by recombinant DNA techniques in which immunoglobulin heavy and light chain fragments are linked to the Fv region via an engineered span of amino acids. Various methods of generating single chain antibodies are known, including those described in U.S. Pat. No. 4,694,778; Bird (1988) Science 242:423-442; Huston et al. (1988) Proc. Natl. Acad. Sci. USA 85:5879-5883; Ward et al. (1989) Nature 334:54454; Skerra et al. (1988) Science 242:1038-1041.

Specifically binds: As used herein, the term “specifically binds,” with respect to an antigen binding domain, such as an antibody agent or a portion of a chimeric antigen receptor, refers to an antigen binding domain or antibody agent which recognizes a specific antigen, but does not substantially recognize or bind other molecules in a sample. For example, an antigen binding domain or antibody agent that specifically binds to an antigen from one species may also bind to that antigen from one or more species. But, such cross-species reactivity does not itself alter the classification of an antigen binding domain or antibody agent as specific. In another example, an antigen binding domain or antibody agent that specifically binds to an antigen may also bind to different allelic forms of the antigen. However, such cross reactivity does not itself alter the classification of an antigen-binding domain or antibody agent as specific. In some instances, the terms “specific binding” or “specifically binding,” can be used in reference to the interaction of an antigen binding domain or antibody agent, a protein, or a peptide with a second chemical species, to mean that the interaction is dependent upon the presence of a particular structure (e.g., an antigenic determinant or epitope) on the chemical species; for example, an antigen binding domain or antibody agent recognizes and binds to a specific protein structure rather than to proteins generally. If an antigen binding domain or antibody agent is specific for epitope “A”, the presence of a molecule containing epitope A (or free, unlabeled A), in a reaction containing labeled “A” and the antigen binding domain or antibody agent, will reduce the amount of labeled A bound to the antibody.

Subject: As used herein, the term “subject” refers to an organism, for example, a mammal (e.g., a human, a non-human mammal, a non-human primate, a primate, a laboratory animal, a mouse, a rat, a hamster, a gerbil, a cat, or a dog). In some embodiments, a human subject is an adult, adolescent, or pediatric subject. In some embodiments, a subject is suffering from a disease, disorder or condition, e.g., a disease, disorder, or condition that can be treated as provided herein, e.g., a cancer or a tumor listed herein. In some embodiments, a subject is susceptible to a disease, disorder, or condition; in some embodiments, a susceptible subject is predisposed to and/or shows an increased risk (as compared to the average risk observed in a reference subject or population) of developing the disease, disorder, or condition. In some embodiments, a subject displays one or more symptoms of a disease, disorder, or condition. In some embodiments, a subject does not display a particular symptom (e.g., clinical manifestation of disease) or characteristic of a disease, disorder, or condition. In some embodiments, a subject does not display any symptom or characteristic of a disease, disorder, or condition. In some embodiments, a subject is a patient. In some embodiments, a subject is an individual to whom diagnosis and/or therapy is and/or has been administered.

Target: As used herein, the term “target” refers to a cell, tissue, organ, or site within the body that is the subject of provided methods, systems, and/or compositions, for example, a cell, tissue, organ or site within a body that is in need of treatment or is preferentially bound by, for example, a CAR, as described herein.

T cell receptor: As used herein, the term “T cell receptor” or “TCR” refers to a complex of membrane proteins that participate in the activation of T cells (also referred to interchangeably as T-lymphocytes) in response to the presentation of antigen. A TCR is responsible for recognizing antigens bound to major histocompatibility complex molecules. A TCR comprises a heterodimer of an alpha (α) and beta (β) chain, although in some cells the TCR comprises gamma and delta (γ/δ) chains. TCRs may exist in alpha/beta and gamma/delta forms, which are structurally similar but have distinct anatomical locations and functions. Each chain comprises two extracellular domains, a variable and constant domain. In some embodiments, a TCR may be modified on any cell comprising a TCR, including, for example, a helper T cell, a cytotoxic T cell, a memory T cell, regulatory T cell, natural killer T cell, and gamma delta T cell. In some embodiments, the T-lymphocytes are alpha/beta T lymphocytes. In some embodiments, the T-lymphocytes are gamma/delta T lymphocytes.

Therapeutic: As used herein, the term “therapeutic” refers to a treatment and/or prophylaxis. A therapeutic effect is obtained by suppression, remission, or eradication of a disease state.

Transfected: As used herein, the term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

Transgene: As used herein, the term “transgene” refers to an exogenous nucleic acid sequence comprised in a cell, e.g., in the genome of the cell, in which the nucleic acid sequence does not naturally occur. In some embodiments, a transgene may comprise or consist of a nucleic acid sequence encoding a gene product, e.g., a CAR. In some embodiments, a transgene may comprise or consist of an expression construct, e.g., a nucleic acid sequence encoding a gene product under the control of a regulatory element, e.g., a promoter.

Treat: As used herein, the term “treat,” “treatment,” or “treating” refers to partial or complete alleviation, amelioration, delay of onset of, inhibition, prevention, relief, and/or reduction in incidence and/or severity of one or more symptoms or features of a disease, disorder, and/or condition. In some embodiments, treatment may be administered to a subject who does not exhibit signs or features of a disease, disorder, and/or condition (e.g., may be prophylactic). In some embodiments, treatment may be administered to a subject who exhibits only early or mild signs or features of the disease, disorder, and/or condition, for example for the purpose of decreasing the risk of developing pathology associated with the disease, disorder, and/or condition. In some embodiments, treatment may be administered to a subject who exhibits established, severe, and/or late-stage signs of the disease, disorder, or condition. In some embodiments, treating may comprise administering to a subject an immune cell comprising a genetically engineered cell expressing a CAR (e.g., a T lymphocyte, B-lymphocyte, NK cell) or administering to a subject a hematopoietic stem cell transplant comprising genetically engineered stem cells.

Tumor: As used herein, the term “tumor” refers to an abnormal growth of cells or tissue. In some embodiments, a tumor may comprise cells that are precancerous (e.g., benign), malignant, pre-metastatic, metastatic, and/or non-metastatic. In some embodiments, a tumor is associated with, or is a manifestation of, a cancer. In some embodiments, a tumor may be a disperse tumor or a liquid tumor. In some embodiments, a tumor may be a solid tumor.

### Genetically Engineered Cells

Some aspects of this disclosure provide cells, e.g., mobilized lymphocytes or descendants thereof, e.g., T-lymphocytes, B-lymphocytes, NK cells, comprising a heterologous nucleic acid comprising a transgene encoding a CAR. In some embodiments, the cell or a descendant thereof is a lymphocyte obtained from a subject after hematopoietic stem cell mobilization.

In some embodiments, the transgene encodes a chimeric antigen receptor targeting a human antigen associated with a disease or disorder, e.g., targeting lineage-specific cell-surface antigen associated with a hyperproliferative disease. In some embodiments, the CAR is operably linked to a cell type specific promoter or a constitutive promoter. In some embodiments, the cell type specific promoter is a CD8 promoter. In some embodiments, the cells type-specific promoter is a CD3delta promoter. In some embodiments, the cell type-specific promoter is a CD56 promoter. In some embodiments, the cell type-specific promoter is a CD244 promoter. In some embodiments, the cell type-specific promoter is a CD94 promoter. In some embodiments, the cell type-specific promoter is an NKG2D promoter.

In some embodiments, the transgene encodes a chimeric antigen receptor comprising a binding domain, a hinge domain, a transmembrane domain, at least one co-stimulatory domain, a cytoplasmic signaling domain, or a combination thereof. In some embodiments, the binding domain comprises an antibody, or an antigen-binding antibody fragment, that binds an antigen. In some embodiments, the binding domain comprises an scFv or a single domain antibody that binds to an antigen. In some embodiments, the antigen is a lineage-specific cell-surface antigen. In some embodiments, expression of the antigen is associated with a hyperproliferative disease. In some embodiments, the hyperproliferative disease is a hematopoietic malignancy, such as a myeloid malignancy or a lymphoid malignancy. In some embodiments, the lineage-specific cell-surface antigen CD33, CD123, CD19, CLL-1, CD30, CD38, BCMA, CD5, CD6, CD7, or any other lineage-specific cell surface antigen, such as those described herein. In some embodiments, the hinge domain of the CAR is a CD8α (CD8alpha) hinge domain. In some embodiments, the transmembrane domain of the

CAR is a CD8 or CD28 transmembrane domain. In some embodiments, the costimulatory domain of the CAR is a 4-1BB or CD28 costimulatory domain, or a combination thereof. In some embodiments, the cytoplasmic signaling domain of the CAR is a CD3ζ (CD3zeta) cytoplasmic signaling domain.

In some embodiments, the cells are lymphocyte cells. In some embodiments, the cells are T-cells, also referred to as T lymphocytes. In some embodiments, the cells are alpha/beta T-cells. In some embodiments, the cells are gamma/delta T-cells. In some embodiments, the cells are natural killer (NK) cells. In some embodiments, the cells are natural killer T-cells (NKT cells). In some embodiments, a T lymphocyte is a naïve T lymphocyte, an effector T lymphocyte, a memory T lymphocyte, or a regulatory T lymphocyte. In some embodiments, the cells are B cells, also referred to as B lymphocytes.

In some embodiments, the immune effector cells provided herein are T lymphocytes, which may be characterized by expression of CD3 (i.e., CD3+). In some embodiments, the T cells express the T cell receptor (TCR) α and β chains. In some embodiments, the T cells express the TCR γ and δ chains. T-lymphocytes may be further classified based on expression of other cell surface markers, for example CD4 or CD8. Expression of CD4 or CD8 broadly classifies T cells as “T helper cells” characterized by expression of CD4 (CD4+) or “cytotoxic T cells” characterized by expression of CD8 (CD8+). As will be appreciated by one of ordinary skill in the art, CD4+ T cells and CD8+ T cells have distinct cellular functions. In some embodiments, the T cells are CD4+ T cells, which may be further distinguished as Th1 or Th2 CD4+cells. In some embodiments, the T cells are CD8+ T cells (also referred to as cytotoxic T lymphocytes, CTL; CD8 T cells).

In some embodiments, the T-cells are cytotoxic T cells. In some embodiments, the T-cells are central memory T cells (TCM), stem memory T cells (TSCM), stem-cell-like memory T cells (or stem-like memory T cells), and effector memory T cells, for example, TEM cells and TEMRA (CD45RA+) cells, effector T cells, or T helper cells, e.g., Th1 cells, Th2 cells, Th9 cells, Th17 cells, Th22 cells, Tfh (follicular helper) cells, T regulatory cells (Tregs, FoxP3+ T cells), natural killer T cells (NKT cells), mucosal associated invariant T cells (MAIT), and γδ T cells. In some embodiments, the T cell expresses a cell death ligand, such as PD1. In some embodiments, T cells, such as CD8+ T cells that express PD1 are considered “exhausted” T cells and promote immune suppression.

In some embodiments, the immune effector cells provided herein are T lymphocytes, which may be characterized by expression of CD19 (i.e., CD19+). In some embodiments, the immune effector cells provided herein are NK cells, which may be characterized by expression of CD56 (i.e., CD56+).

In some embodiments, a cell of the disclosure is a mobilized lymphocyte. In some embodiments, cells for use in the methods of the disclosure, e.g., mobilized lymphocytes, are collected from a subject who has undergone hematopoietic stem cell mobilization. In some embodiments, a cell of the disclosure is a peripheral blood mononuclear cell (PBMC), e.g., a lymphocyte, obtained from a subject who has undergone hematopoietic stem cell mobilization. Hematopoietic stem cell mobilization is a process in which subjects are exposed to mobilizing agents which promote mobilization of cells (e.g., hematopoietic stem cells), normally localized in the bone marrow, to migrate into the peripheral circulatory system, allowing the stem cells to be harvested by taking a blood sample, e.g., an apheresis sample. See, e.g., Karpova D, Rettig M P, DiPersio J F. Mobilized peripheral blood: an updated perspective. F1000Res. 2019;8:F1000 Faculty Rev-2125. Published 2019 Dec. 20. doi:10.12688/f1000research.21129.1. In some embodiments, hematopoietic stem cell mobilization comprises administering to the subject one or more of etoposide, plerixafor, cyclophosphamide, and/or granulocyte colony-stimulating factor (G-CSF). In some embodiments, hematopoietic stem cell mobilization comprises expansion of the hematopoietic stem cell population in the subject. In some embodiments, a blood sample (e.g., apheresis sample) collected from a subject who has undergone hematopoietic stem cell mobilization comprises different relative proportions of types of cells (e.g., relative to the total cell count) compared to a blood sample (e.g., apheresis sample) collected from a steady state subject who has not undergone hematopoietic stem cell mobilization.

In some embodiments, a blood sample from a subject who has undergone hematopoietic stem cell mobilization may supply mobilized lymphocytes (e.g., for generating a genetically engineered cell comprising a heterologous nucleic acid encoding a CAR targeting lineage-specific cell-surface antigen associated with a hyperproliferative disease) and HSCs (e.g., for generating a genetically engineered HSC as described herein). Methods of hematopoietic stem cell mobilization, and obtaining HSCs are described, e.g., in PCT Application No. US2016/057339, which is herein incorporated by reference in its entirety.

In some embodiments, the mammalian subject is a non-human primate, a rodent (e.g., mouse or rat), a bovine, a porcine, an equine, or a domestic animal. In some embodiments, the mobilized lymphocytes and/or HSCs are obtained from a human subject, such as a human subject having a hematopoietic malignancy. In some embodiments, the mobilized lymphocytes and/or HSCs are obtained from a healthy donor. In some embodiments, the mobilized lymphocytes and/or HSCs are obtained from the subject to whom the immune cells expressing the chimeric receptors will be subsequently administered. Mobilized lymphocytes that are administered to the same subject from which the cells were obtained are referred to as autologous cells, whereas mobilized lymphocytes that are obtained from a subject who is not the subject to whom the cells will be administered are referred to as allogeneic cells.

Specific populations of cells or cell types may be isolated from a population of mobilized lymphocyte cells. In some embodiments, T cells are isolated by methods well known in the art, including commercially available isolation methods (see, for example, Rowland-Jones et al., Lymphocytes: A Practical Approach, Oxford University Press, New York (1999)). T cell separation and isolation methods are well known in the art, e.g., T cells can be isolated using various cell surface markers or combinations of markers (including positive and negative selection) depending on the desired T-cell subtype, including but not limited to, CD3, CD4, CD8, CD34 (for hematopoietic stem and progenitor cells) and the like, can be used to separate the cells, as is well known in the art (see Kearse, T Cell Protocols: Development and Activation, Humana Press, Totowa N.J. (2000); De Libero, T Cell Protocols, Vol. 514 of Methods in Molecular Biology, Humana Press, Totowa N.J. (2009); Su et al., Methods Mol. Biol. 806:287-299 (2012); Bluestone et al., Sci. Transl. Med. 7(315) (doi: 10.1126/scitranslmed.aad4134)(2015); Miyara et al., Nat. Rev. Rheumatol. 10:543-551 (2014); Liu et al., J. Exp. Med. 203:1701-1711 (2006); Seddiki et al., J. Exp. Med. 203:1693-1700 (2006); Ukena et al., Exp. Hematol. 39:1152-1160 (2011); Chen et al., J. Immunol. 183:4094-4102 (2009); Putnam et al., Diabetes 58:652-662 (2009); Putnam et al., Am. Tranplant. 13:3010-3020 (2013); Lee et al., Cancer Res. 71:2871-2881 (2011); MacDonald et al., J Clin. Invest. 126:1413-1424 (2016)). Methods for isolating and expanding regulatory T cells are also commercially available (see, for example, BD Biosciences, San Jose, Calif.; STEMCELL Technologies Inc., Vancouver, Canada; eBioscience, San Diego, Calif.; Invitrogen, Carlsbad, Calif.).

In some embodiments, the genetically engineered cells (or descendants thereof) are autologous to a subject to which they are administered back, e.g., after being contacted with a heterologous nucleic acid encoding a CAR. In some embodiments, the cells are non-autologous, e.g., allogeneic to a subject to which they are administered, e.g., after being contacted with a heterologous nucleic acid encoding a CAR.

In some embodiments, mobilized cells (e.g., mobilized lymphocytes) are obtained from a subject, genetically engineered to express a CAR, and then administered back to the same subject. In some embodiments, mobilized cells (e.g., mobilized lymphocytes) are obtained from a subject, genetically engineered to express a CAR, and then administered to a different subject, e.g., an HLA-matched subject.

Any suitable method for isolating particular cell types (e.g., T-lymphocytes, B-lymphocytes, NK cells) from a population of cells (e.g., mobilized PBMCs) that can be used for recombinant expression of a CAR can be used, including, but not limited to, methods known in the art, e.g., those described in Sadelain et al., Nat. Rev. Cancer 3:35-45 (2003); Morgan et al., Science 314:126-129 (2006), Panelli et al., J Immunol. 164:495-504 (2000); Panelli et al., J Immunol. 164:4382-4392 (2000)), Dupont et al., Cancer Res. 65:5417-5427 (2005); Papanicolaou et al., Blood 102:2498-2505 (2003)).

Delivery of the heterologous nucleic acid encoding a CAR targeting a lineage-specific cell-surface antigen provided herein to cells can be via any suitable method or technology. For example, viral vector systems are particularly suitable for introducing nucleic acids to mature cells, but other delivery modalities can be used as well. Other methods of creating targeted integrations are known in the art, and include, without limitation, the use of transposons or viral vectors (e.g., AAV vectors) that exhibit site specificity or site preference for integrating into the genome of a host cell. While some exemplary methods of delivery of cell-type specific inducible expression systems are provided herein, additional suitable systems will be apparent to the skilled artisan based on the present disclosure and the knowledge in the art. The disclosure is not limited in this respect.

### Expression Constructs

Expression constructs suitable for expressing chimeric antigen receptors in effector lymphocytes, e.g., in T-lymphocytes, B-lymphocytes, or NK cells are well known in the art. Some exemplary expression constructs are provided herein, and the skilled artisan will be aware of additional suitable constructs. Typically, a suitable expression construct will comprise a nucleic acid sequence encoding the CAR under the control of a heterologous promoter. In some embodiments, the promoter is a constitutively active promoter. In some embodiments, the promoter is a cell-type-specific promoter. In some embodiments, the promoter is an inducible promoter. In some embodiments, the expression construct comprises additional elements, such as, for example, a polyadenylation signal, a 5′ UTR and/or a 3′ UTR sequence, an intron sequence. Depending on the delivery route of the expression construct to the target cells, e.g. to the lymphocytes, the expression construct may comprise additional elements, e.g., viral packaging and integration elements in case of viral delivery.

Some aspects of this disclosure provide heterologous nucleic acids and expression constructs thereof useful for expressing a transgene, e.g., a CAR, in a specific cell type. For example, some aspects of this disclosure provide expression constructs that are only active in specific cell types or sub-types (e.g., T-lymphocytes, pre-T cells, mature T-lymphocytes, NK cells, B-lymphocytes, etc.). In some embodiments, cell type specific and inducible expression is achieved by expressing an inducible expression system, in a cell type-specific manner, e.g., by placing one of the components of the inducible system under the control of a regulatory element, e.g., a promoter, that is only active in the respective cell type.

In some embodiments, a promoter for expressing a CAR is under control of a cell-type specific promoter, which is a promoter that is expressed in one specific cell type, e.g., in T-lymphocytes, B-lymphocytes, NK cells, or in a specific sub-group of cells, e.g., in lymphocytes, but not in cells other than the specific cell type.

In some embodiments, the CAR comprises an antigen binding domain that binds to an antigen that is associated with a disease or disorder, e.g., with a hyperproliferative disease or disorder.

In some embodiments, the CAR is a first generation CAR. In some embodiments, the CAR is a second generation CAR. In some embodiments, the CAR is a third generation CAR. In some embodiments, the CAR is a fourth or fifth generation CAR, or an armored CAR. Exemplary CAR constructs are provided herein, and additional suitable CAR constructs will be apparent to the skilled artisan based on the present disclosure and the knowledge in the art. For an illustration of various CAR backbones or frameworks that are suitable for use in connection with the presently provided expression systems, see, the following, exemplary, and non-limiting publications: Sadelain et al., Cancer Discov. 3(4):388-398 (2013); Jensen et al., Immunol. Rev. 257:127-133 (2014); Sharpe et al., Dis. Model Mech. 8(4):337-350 (2015); Brentjens et al., Clin. Cancer Res. 13:5426-5435 (2007); Gade et al., Cancer Res. 65:9080-9088 (2005); Maher et al., Nat. Biotechnol. 20:70-75 (2002); Kershaw et al., J. Immunol. 173:2143-2150 (2004); Sadelain et al., Curr. Opin. Immunol. (2009); Hollyman et al., J. Immunother. 32:169-180 (2009)).

First generation CARs are typically composed of an extracellular antigen binding domain, for example, a single-chain variable fragment (scFv), fused to a transmembrane domain, which is fused to a cytoplasmic/intracellular domain of the T cell receptor chain. Typically, first generation CARs comprise the intracellular domain of CD3ζ, which transmits signals from endogenous T cell receptors (TCRs) “First generation” CARs can provide de novo antigen recognition and cause activation of both CD4+ and CD8+ T cells through their CD3ζ chain signaling domain in a single fusion molecule, independent of HLA-mediated antigen presentation.

Second-generation CARs comprise an antigen-binding domain fused to an intracellular signaling domain capable of activating T cells and a co-stimulatory domain designed to augment T cell potency and persistence. See, e.g., Sadelain et al., Cancer Discov. 3:388-398, 2013. Second generation CARs comprise an intracellular co-stimulatory domain in addition to the CD3ζ domain, for example, a CD28, 4-1BB, ICOS, OX40, or similar co-stimulatory domain. Thus, second generation CARs provide both co-stimulation, for example, by CD28 or 4-1BB domains, and activation, for example, by a CD3ζ signaling domain. Second Generation CARs may improve the anti-tumor activity of T cells as compared to first generation CARs.

Third generation CARs comprise more than one co-stimulatory domains, for example, two costimulatory domains, e.g., both a CD28 and a 4-1BB domain, and an activation domain, for example, by comprising a CD3ζ activation domain.

In general, CARs comprise an extracellular antigen binding domain, a transmembrane domain and an intracellular domain. Typically, the antigen binding domain binds to an antigen of interest, such as an antigen associated with a disease or disorder, e.g., with a neoplastic or malignant disease or disorder. In some embodiments, the antigen-binding domain is an antibody or an antigen-binding fragment thereof. In some embodiments, the antigen binding domain is a single chain antibody or antigen-binding fragment thereof. In some embodiments, the antigen-binding domain comprises an scFv. In some embodiments, the antigen-binding domain comprises a single domain antibody, e.g., a camelid antibody, or a humanized derivative thereof. In some embodiments, the antigen-binding domain comprises a receptor or a receptor ligand.

Some exemplary CARs for use in the expression systems disclosed herein are provided herein. Additional CARs will be apparent to the skilled artisan based on the present disclosure in view of the knowledge in the art regarding the design of CARs, e.g., as illustrated in Sadelain et al., Cancer Discov. 3(4):388-398 (2013); Jensen et al., Immunol. Rev. 257:127-133 (2014); Sharpe et al., Dis. Model Mech. 8(4):337-350 (2015), and references cited therein.

In some embodiments, the CAR for use in the present invention comprises an extracellular domain that includes an antigen binding domain that binds to a lineage-specific cell-surface antigen, e.g., associated with a hyperproliferative disease. In some embodiments, the antigen binding domain binds to a lineage-specific cell-surface antigen expressed on the surface of a neoplastic cell or a malignant cell. In some embodiments, the antigen binding domain binds to a lineage-specific cell-surface antigen expressed or present on the surface of a pathogenic or pathologic cell, e.g., a cell that has been infected by an infectious agent, or a cell that is characterized by a pathogenic or pathologic state. In some embodiments, the antigen binding domain can be an scFv or a Fab, a single domain antibody, or any suitable antigen binding fragment of an antibody (see Sadelain et al., Cancer Discov. 3:38-398 (2013)).

In some embodiments, the antigen binding domain comprises a sequence of a human, a humanized, a chimeric, or a CDR-grafted antibody, or antigen-binding antibody fragment. In some embodiments, the antigen binding domain comprises an scFv. Exemplary scFvs are provided herein, and additional suitable scFvs will be apparent to the skilled artisan based on the present disclosure and the knowledge in the art related to scFvs (see, for example, Huston, et al., Proc. Nat. Acad. Sci. USA 85:5879-5883 (1988); Ahmad et al., Clin. Dev. Immunol. 2012: ID980250 (2012); U.S. Pat. Nos. 5,091,513, 5,132,405 and 4,956,778; and U.S. Patent Publication Nos. 20050196754 and 20050196754)). The disclosure is not limited in this aspect.

Alternatively to using an antigen binding domain derived from an antibody, a CAR extracellular domain can comprise a ligand or extracellular ligand binding domain of a receptor (see Sadelain et al., Cancer Discov. 3:388-398 (2013); Sharpe et al., Dis. Model Mech. 8:337-350 (2015)).

In some embodiments, the CAR binds to an antigen expressed on malignant cells, which is also referred to sometimes as a cancer antigen. Any CAR targeting a suitable cancer antigen can be used in the context of the presently disclosed expression systems. Exemplary cancer antigens and exemplary cancers are provided below:

In some embodiments, the lineage-specific cell-surface antigen chosen from: CD5, CD6, CD7, CD13, CD19, CD22, CD20,CD25, CD30, CD32, CD38, CD44, CD45, CD47, CD56, 96, CD117, CD123, CD135,CD174, CLL-1, BCMA, folate receptor β, IL1RAP, MUC1, NKG2D/NKG2DL, TIM-3, or WT1.

In some embodiments, the lineage-specific cell-surface antigen chosen from: CD1a, CD1b, CD1c, CD 1d, CD1e, CD2, CD3, CD3d, CD3e, CD3g, CD4, CD5, CD6, CD7, CD8a, CD8b, CD9, CD10, CD11a, CD11b, CD11c, CD11d, CDw12, CD13, CD14, CD15, CD16, CD16b, CD17, CD18, CD19, CD20, CD21, CD22, CD23, CD24, CD25, CD26, CD27, CD28, CD29, CD30, CD31, CD32a, CD32b, CD32c, CD34, CD35, CD36, CD37, CD38, CD39, CD40, CD41, CD42a, CD42b, CD42c, CD42d, CD43, CD44, CD45, CD45RA, CD45RB, CD45RC, CD45RO, CD46, CD47, CD48, CD49a, CD49b, CD49c, CD49d, CD49e, CD49f, CD50, CD51, CD52, CD53, CD54, CD55, CD56, CD57, CD58, CD59, CD60a, CD61, CD62E, CD62L, CD62P, CD63, CD64a, CD65, CD65s, CD66a, CD66b, CD66c, CD66F, CD68, CD69, CD70, CD71, CD72, CD73, CD74, CD75, CD75S, CD77, CD79a, CD79b, CD80, CD81, CD82, CD83, CD84, CD85A, CD85C, CD85D, CD85E, CD85F, CD85G, CD85H, CD85I, CD85J, CD85K, CD86, CD87, CD88, CD89, CD90, CD91, CD92, CD93, CD94, CD95, CD96, CD97, CD98, CD99, CD99R, CD100, CD101, CD102, CD103, CD104, CD105, CD106, CD107a, CD107b, CD108, CD109, CD110, CD111, CD112, CD113, CD114, CD115, CD116, CD117, CD118, CD119, CD120a, CD120b, CD121a, CD121b, CD121a, CD121b, CD122, CD123, CD124, CD125, CD126, CD127, CD129, CD130, CD131, CD132, CD133, CD134, CD135, CD136, CD137, CD138, CD139, CD140a, CD140b, CD141, CD142, CD143, CD14, CDw145, CD146, CD147, CD148, CD150, CD152, CD152, CD153, CD154, CD155, CD156a, CD156b, CD156c, CD157, CD158b1, CD158b2, CD158d, CD158e1/e2, CD158f, CD158g, CD158h, CD158i, CD158j, CD158k, CD159a, CD159c, CD160, CD161, CD163, CD164, CD165, CD166, CD167a, CD168, CD169, CD170, CD171, CD172a, CD172b, CD172g, CD173, CD174, CD175, CD175s, CD176, CD177, CD178, CD179a, CD179b, CD180, CD181, CD182, CD183, CD184, CD185, CD186, CD191, CD192, CD193, CD194, CD195, CD196, CD197, CDw198, CDw199, CD200, CD201, CD202b, CD203c, CD204, CD205, CD206, CD207, CD208, CD209, CD210a, CDw210b, CD212, CD213a1, CD213a2, CD215, CD217, CD218a, CD218b, CD220, CD221, CD222, CD223, CD224, CD225, CD226, CD227, CD228, CD229, CD230, CD231, CD232, CD233, CD234, CD235a, CD235b, CD236, CD236R, CD238, CD239, CD240, CD241, CD242, CD243, CD244, CD245, CD246, CD247, CD248, CD249, CD252, CD253, CD254, CD256, CD257, CD258, CD261, CD262, CD263, CD264, CD265, CD266, CD267, CD268, CD269, CD270, CD272, CD272, CD273, CD274, CD275, CD276, CD277, CD278, CD279, CD280, CD281, CD282, CD283, CD284, CD286, CD288, CD289, CD290, CD292, CDw293, CD294, CD295, CD296, CD297, CD298, CD299, CD300a, CD300c, CD300e, CD301, CD302, CD303, CD304, CD305, CD306, CD307a, CD307b, CD307c, CD307d, CD307e, CD309, CD312, CD314, CD315, CD316, CD317, CD318, CD319, CD320, CD321, CD322, CD324, CD325, CD326, CD327, CD328, CD329, CD331, CD332, CD333, CD334, CD335, CD336, CD337, CD338, CD339, CD340, CD344, CD349, CD350, CD351, CD352, CD353, CD354, CD355, CD357, CD358, CD359, CD360, CD361, CD362 or CD363.

In some embodiments, the lineage-specific cell-surface antigen is selected from those listed below: CD5, CD6, CD7, CD10, CD19, CD20, CD22, CD30, CD33, CD34, CD38, CD41, CD44, CD49f, CD56, CD74, CD123, CD133, CD138, mesothelin (MSLN), prostate specific membrane antigen (PSMA), prostate stem cell antigen (PCSA), carbonic anhydrase IX (CAIX), carcinoembryonic antigen (CEA), epithelial glycoprotein2 (EGP 2), epithelial glycoprotein-40 (EGP-40), epithelial cell adhesion molecule (EpCAM), folate-binding protein (FBP), fetal acetylcholine receptor (AChR), folate receptor-α and β (FRα and β), Ganglioside G2 (GD2), Ganglioside G3 (GD3), human Epidermal Growth Factor Receptor 2 (HER-2/ERB2), Epidermal Growth Factor Receptor vIII (EGFRvIII), ERB3, ERB4, human telomerase reverse transcriptase (hTERT), Interleukin-13 receptor subunit alpha-2 (IL-13Rα2), κ-light chain, kinase insert domain receptor (KDR), Lewis A (CA19.9), Lewis Y (LeY), L1 cell adhesion molecule (L1CAM), melanoma-associated antigen 1 (melanoma antigen family A1, MAGE-A1), Mucin 16 (Muc-16), Mucin 1 (Muc-1), NKG2D ligands, cancer-testis antigen NY-ESO-1, oncofetal antigen (h5T4), tumor-associated glycoprotein 72 (TAG-72), vascular endothelial growth factor R2 (VEGF-R2), Wilms tumor protein (WT-1), type 1 tyrosine-protein kinase transmembrane receptor (ROR1), B7-H3 (CD276), B7-H6 (Nkp30), Chondroitin sulfate proteoglycan-4 (CSPG4), DNAX Accessory Molecule (DNAM-1), Ephrin type A Receptor 2 (EpHA2), Fibroblast Associated Protein (FAP), Gp100/HLA-A2, Glypican 3 (GPC3), HA-1H, HERK-V, IL-11Rα, Latent Membrane Protein 1 (LMP1), Neural cell-adhesion molecule (N-CAM/CD56), and Trail Receptor (TRAIL R). It is understood that these or other cancer antigens can be utilized for targeting by a cancer antigen CAR. While some exemplary suitable CARs and some exemplary suitable CAR target antigens are disclosed herein, additional suitable CARS and CAR target antigens will be apparent to the skilled artisan based on the present disclosure in view of the knowledge in the art regarding CARs and CAR antigens. See, e.g., International PCT Application PCT/US2017/027601, the entire contents of which are incorporated herein by reference. In some embodiments, a CAR binds to a lineage-specific cell-surface antigen disclosed herein.

### Genetically Engineered Hematopoietic Stem Cells

Some aspects of this disclosure provide genetically engineered hematopoietic stem cells that have been genetically edited to have reduced expression or loss of expression of a lineage-specific cell-surface antigen, or expression of a variant form of a lineage-specific cell-surface antigen that is not recognized by an CAR targeting the lineage-specific cell-surface. In some embodiments, the genetically engineered stem cells are administered to a subject in the form of a hematopoietic stem cell transplant.

In some embodiments, a genetically engineered stem cell provided herein comprises a genomic modification that results in a loss of expression of a lineage-specific cell-surface antigen, or expression of a variant form of the lineage-specific cell-surface antigen that is not recognized by a CAR targeting the lineage-specific cell-surface antigen, and does not comprise an expression construct that encodes an exogenous protein, e.g., does not comprise an expression construct encoding a CAR.

In some embodiments, a genetically engineered hematopoietic stem cell provided herein expresses substantially no lineage-specific cell-surface antigen protein, e.g., expresses no lineage-specific cell-surface antigen protein that can be measured by a suitable method, such as an immunostaining method. In some embodiments, a genetically engineered hematopoietic stem cell provided herein expresses substantially no wild-type a lineage-specific cell-surface antigen protein, but expresses a mutant lineage-specific cell-surface antigen variant, e.g., a variant not recognized by an immunotherapeutic agent targeting the lineage-specific cell-surface antigen, e.g., a CAR-expressing cell, or an antibody, antibody fragment, or antibody-drug conjugate (ADC) targeting a lineage-specific cell-surface antigen. In some embodiments, the lineage-specific cell-surface antigen is any one of the lineage-specific cell-surface antigens described herein.

In some embodiments, the genetically engineered hematopoietic stem cells provided herein are hematopoietic cells, e.g., hematopoietic stem cells. Hematopoietic stem cells (HSCs) are typically capable of giving rise to both myeloid and lymphoid progenitor cells that further give rise to myeloid cells (e.g., monocytes, macrophages, neutrophils, basophils, dendritic cells, erythrocytes, platelets, etc.) and lymphoid cells (e.g., T cells, B cells, NK cells), respectively. HSCs are characterized by the expression of the cell surface marker CD34 (e.g., CD34+), which can be used for the identification and/or isolation of HSCs, and absence of cell surface markers associated with commitment to a cell lineage.

In some embodiments, a population of genetically engineered hematopoietic stem cells described herein comprises a plurality of genetically engineered hematopoietic stem cells. In some embodiments, a population of genetically engineered hematopoietic stem cells described herein comprises a plurality of genetically engineered hematopoietic progenitor cells. In some embodiments, a population of genetically engineered stem cells described herein comprises a plurality of genetically engineered hematopoietic stem cells and a plurality of genetically engineered hematopoietic progenitor cells.

In some embodiments, the genetically engineered HSCs are obtained from a subject, such as a human subject. Methods of obtaining HSCs are described, e.g., in PCT Application No. US2016/057339, which is herein incorporated by reference in its entirety. In some embodiments, the HSCs are mobilized from the bone marrow of the subject by administration of a mobilization agent, e.g., etoposide, plerixafor, cyclophosphamide, and/or granulocyte colony-stimulating factor (G-CSF). In some embodiments, the HSCs are obtained from a human subject, such as a human subject having a hematopoietic malignancy. In some embodiments, the HSCs are obtained from a healthy donor. In some embodiments, the HSCs are obtained from the same subject from whom the mobilized lymphoctyes are obtained. In some embodiments, the HSCs are obtained from the subject to whom the genetically engineered cells expressing the CARs will be subsequently administered. In some embodiments, the HSCs are obtained from a subject that is not the subject to whom the genetically engineered cells expressing the CARs will be subsequently administered. HSCs that are administered to the same subject from which the cells were obtained are referred to as autologous cells, whereas HSCs that are obtained from a subject who is not the subject to whom the cells will be administered are referred to as allogeneic cells.

In some embodiments, a population of genetically engineered hematopoietic stem cells is a heterogeneous population of cells, e.g. heterogeneous population of genetically engineered hematopoietic stem cells containing different mutations in the lineage-specific cell-surface antigen. In some embodiments, at least 40%, at least 50%, at least 60%, at least 70%, at least 75%, at least 80%, at least 85%, at least 90%, or at least 95% of copies of a gene encoding the lineage-specific cell-surface antigen in the population of genetically engineered hematopoietic stem cells comprise a mutation effected by a genome editing approach described herein, e.g., by a CRISPR/Cas system using a gRNA provided herein.

In some embodiments, a genetically engineered hematopoietic cell, such as, for example, a genetically engineered hematopoietic stem or progenitor cell or a genetically engineered immune effector cell) described herein is generated via genome editing technology, which includes any technology capable of introducing targeted changes, also referred to as “edits,” into the genome of a cell.

Methods of genetically engineering hematopoietic stem and/or progenitor cells and immune effector cells will evident to one of ordinary skill in the art. For example, exemplary methods of genetically engineering hematopoietic stem and/or progenitor cells are provided in PCT Publication Nos. WO 2017/066760 and WO 2020/047164. One exemplary suitable genome editing technology is “gene editing,” comprising the use of a RNA-guided nuclease, e.g., a CRISPR/Cas nuclease, to introduce targeted single- or double-stranded DNA breaks in the genome of a cell, which trigger cellular repair mechanisms, such as, for example, nonhomologous end joining (NHEJ), microhomology-mediated end joining (MMEJ, also sometimes referred to as “alternative NHEJ” or “alt-NHEJ”), or homology-directed repair

(HDR) that typically result in an altered nucleic acid sequence (e.g., via nucleotide or nucleotide sequence insertion, deletion, inversion, or substitution) at or immediately proximal to the site of the nuclease cut. See, Yeh et al. Nat. Cell. Biol. (2019) 21: 1468-1478; e.g., Hsu et al. Cell (2014) 157: 1262-1278; Jasin et al. DNA Repair (2016) 44: 6-16; Sfeir et al. Trends Biochem. Sci. (2015) 40: 701-714.

Another exemplary suitable genome editing technology is “base editing,” which includes the use of a base editor, e.g., a nuclease-impaired or partially nuclease-impaired RNA-guided CRISPR/Cas protein fused to a deaminase that targets and deaminates a specific nucleobase, e.g., a cytosine or adenosine nucleobase of a C or A nucleotide, which, via cellular mismatch repair mechanisms, results in a change from a C to a T nucleotide, or a change from an A to a G nucleotide. See, e.g., Komor et al. Nature (2016) 533: 420-424; Rees et al. Nat. Rev. Genet. (2018) 19(12): 770-788; Anzalone et al. Nat. Biotechnol. (2020) 38: 824-844;

Yet another exemplary suitable genome editing technology includes “prime editing,” which includes the introduction of new genetic information, e.g., an altered nucleotide sequence, into a specifically targeted genomic site using a catalytically impaired or partially catalytically impaired RNA-guided nuclease, e.g., a CRISPR/Cas nuclease, fused to an engineered reverse transcriptase (RT) domain. The Cas/RT fusion is targeted to a target site within the genome by a guide RNA that also comprises a nucleic acid sequence encoding the desired edit, and that can serve as a primer for the RT. See, e.g., Anzalone et al. Nature (2019) 576 (7785): 149-157.

The use of genome editing technology typically features the use of a suitable RNA-guided nuclease, which, in some embodiments, e.g., for base editing or prime editing, may be catalytically impaired, or partially catalytically impaired. Examples of suitable RNA-guided nucleases include CRISPR/Cas nucleases. For example, in some embodiments, a suitable RNA-guided nuclease for use in the methods of genetically engineering cells provided herein is a Cas9 nuclease, e.g., an SpCas9 or an SaCas9 nuclease. For another example, in some embodiments, a suitable RNA-guided nuclease for use in the methods of genetically engineering cells provided herein is a Cas12 nuclease, e.g., a Cas12a nuclease. Exemplary suitable Cas12 nucleases include, without limitation, AsCas12a, FnCas12a, other Cas12a orthologs, and Cas12a derivatives, such as the MAD7 system (MAD7™, Inscripta, Inc.), or the Alt-R Cas12a (Cpf1) Ultra nuclease (Alt-R® Cas12a Ultra; Integrated DNA Technologies, Inc.). See, e.g., Gill et al. LIPSCOMB 2017. In United States: Inscripta Inc.; Price et al. Biotechnol. Bioeng. (2020) 117(60): 1805-1816;

In some embodiments, a genetically engineered hematopoietic cell, such as, for example, a genetically engineered hematopoietic stem or progenitor cell or a genetically engineered immune effector cell) described herein is generated by targeting an RNA-guided nuclease, e.g., a CRISPR/Cas nuclease, such as, for example, a Cas9 nuclease or a Cas12a nuclease, to a suitable target site in the genome of the cell, under conditions suitable for the RNA-guided nuclease to bind the target site and cut the genomic DNA of the cell. A suitable RNA-guided nuclease can be targeted to a specific target site within the genome by a suitable guide RNA (gRNA). Suitable gRNAs for targeting CRISPR/Cas nucleases according to aspects of this disclosure are provided herein and exemplary suitable gRNAs are described in more detail elsewhere herein.

In some embodiments, a lineage-specific cell-surface antigen gRNA described herein is complexed with a CRISPR/Cas nuclease, e.g., a Cas9 nuclease. Various Cas9 nucleases are suitable for use with the gRNAs provided herein to effect genome editing according to aspects of this disclosure. Typically, the Cas nuclease and the gRNA are provided in a form and under conditions suitable for the formation of a Cas/gRNA complex, that targets a target site on the genome of the cell, e.g., a target site within the sequence encoding a lineage-specific cell-surface antigen. In some embodiments, a Cas nuclease is used that exhibits a desired PAM specificity to target the Cas/gRNA complex to a desired target domain in the sequence encoding a lineage-specific cell-surface antigen. Suitable target domains and corresponding gRNA targeting domain sequences are provided herein.

In some embodiments, a Cas/gRNA complex is formed, e.g., in vitro, and a target cell is contacted with the Cas/gRNA complex, e.g., via electroporation of the Cas/gRNA complex into the cell. In some embodiments, the cell is contacted with Cas protein and gRNA separately, and the Cas/gRNA complex is formed within the cell. In some embodiments, the cell is contacted with a nucleic acid, e.g., a DNA or RNA, encoding the Cas protein, and/or with a nucleic acid encoding the gRNA, or both.

In some embodiments, genetically engineered cells as provided herein are generated using a suitable genome editing technology, wherein the genome editing technology is characterized by the use of a Cas9 nuclease. In some embodiments, the Cas9 molecule is of, or derived from, Streptococcus pyogenes (SpCas9), Staphylococcus aureus (SaCas9), or Streptococcus thermophilus (stCas9). Additional suitable Cas9 molecules include those of, or derived from, Neisseria meningitidis (NmCas9), Acidovorax avenae, Actinobacillus pleuropneumoniae, Actinobacillus succinogenes, Actinobacillus suis, Actinomyces sp., cycliphilus denitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni (CjCas9), Campylobacter lari, Candidatus Puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, gamma proteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria meningitidis, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tistrella mobilis, Treponema sp., or Verminephrobacter eiseniae. In some embodiments, catalytically impaired, or partially impaired, variants of such Cas9 nucleases may be used. Additional suitable Cas9 nucleases, and nuclease variants, will be apparent to those of skill in the art based on the present disclosure. The disclosure is not limited in this respect.

In some embodiments, the Cas nuclease is a naturally occurring Cas molecule. In some embodiments, the Cas nuclease is an engineered, altered, or modified Cas molecule that differs, e.g., by at least one amino acid residue, from a reference sequence, e.g., the most similar naturally occurring Cas9 molecule or a sequence of Table 50 of PCT Publication No. WO2015/157070, which is herein incorporated by reference in its entirety.

In some embodiments, a Cas nuclease is used that belongs to class 2 type V of Cas nucleases. Class 2 type V Cas nucleases can be further categorized as type V-A, type V-B, type V-C, and type V-U. See, e.g., Stella et al. Nature Structural & Molecular Biology (2017). In some embodiments, the Cas nuclease is a type V-B Cas endonuclease, such as a C2c1. See, e.g., Shmakov et al. Mol Cell (2015) 60: 385-397. In some embodiments, the Cas nuclease used in the methods of genome editing provided herein is a type V-A Cas endonuclease, such as a Cpf1(Cas12a) nuclease. See, e.g., Strohkendl et al. Mol. Cell (2018) 71: 1-9. In some embodiments, a Cas nuclease used in the methods of genome editing provided herein is a Cpf1 nuclease derived from Provetella spp. or Francisella spp., Acidaminococcus sp. (AsCpf1), Lachnospiraceae bacterium (LpCpf1), or Eubacterium rectale. In some embodiments, the Cas nuclease is MAD7.

Both naturally occurring and modified variants of CRISPR/Cas nucleases are suitable for use according to aspects of this disclosure. For example, dCas or nickase variants, Cas variants having altered PAM specificities, and Cas variants having improved nuclease activities are embraced by some embodiments of this disclosure.

Some features of some exemplary, non-limiting suitable Cas nucleases are described in more detail herein, without wishing to be bound to any particular theory.

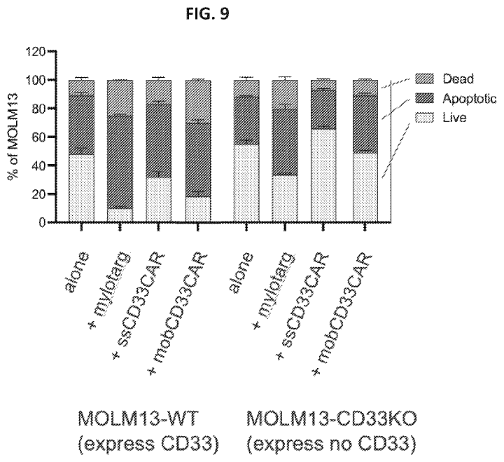

A naturally occurring Cas9 nuclease typically comprises two lobes: a recognition (REC) lobe and a nuclease (NUC) lobe; each of which further comprises domains described, e.g., in PCT Publication No. WO2015/157070, e.g., in FIGS. 9A-9B therein (which application is incorporated herein by reference in its entirety).

The REC lobe comprises the arginine-rich bridge helix (BH), the REC1 domain, and the REC2 domain. The REC lobe appears to be a Cas9-specific functional domain. The BH domain is a long alpha helix and arginine rich region and comprises amino acids 60-93 of the sequence of S. pyogenes Cas9. The REC1 domain is involved in recognition of the repeat:anti-repeat duplex, e.g., of a gRNA or a tracrRNA. The REC1 domain comprises two REC1 motifs at amino acids 94 to 179 and 308 to 717 of the sequence of S. pyogenes Cas9. These two REC1 domains, though separated by the REC2 domain in the linear primary structure, assemble in the tertiary structure to form the REC1 domain. The REC2 domain, or parts thereof, may also play a role in the recognition of the repeat: anti-repeat duplex. The REC2 domain comprises amino acids 180-307 of the sequence of S. pyogenes Cas9.

The NUC lobe comprises the RuvC domain (also referred to herein as RuvC-like domain), the HNH domain (also referred to herein as HNH-like domain), and the PAM-interacting (PI) domain. The RuvC domain shares structural similarity to retroviral integrase superfamily members and cleaves a single strand, e.g., the non-complementary strand of the target nucleic acid molecule. The RuvC domain is assembled from the three split RuvC motifs (RuvC I, RuvCII, and RuvCIII, which are often commonly referred to in the art as RuvCI domain, or N-terminal RuvC domain, RuvCII domain, and RuvCIII domain) at amino acids 1-59, 718-769, and 909-1098, respectively, of the sequence of S. pyogenes Cas9. Similar to the REC1 domain, the three RuvC motifs are linearly separated by other domains in the primary structure, however in the tertiary structure, the three RuvC motifs assemble and form the RuvC domain. The HNH domain shares structural similarity with HNH endonucleases, and cleaves a single strand, e.g., the complementary strand of the target nucleic acid molecule. The HNH domain lies between the RuvC II-III motifs and comprises amino acids 775-908 of the sequence of S. pyogenes Cas9. The PI domain interacts with the PAM of the target nucleic acid molecule and comprises amino acids 1099-1368 of the sequence of S. pyogenes Cas9.

Crystal structures have been determined for naturally occurring bacterial Cas9 nucleases (see, e.g., Jinek et al., Science, 343(6176): 1247997, 2014) and for S. pyogenes Cas9 with a guide RNA (e.g., a synthetic fusion of crRNA and tracrRNA) (Nishimasu et al., Cell, 156:935-949, 2014; and Anders et al., Nature, 2014, doi: 10.1038/nature13579).

In some embodiments, a Cas9 molecule described herein exhibits nuclease activity that results in the introduction of a double strand DNA break in or directly proximal to a target site. In some embodiments, the Cas9 molecule has been modified to inactivate one of the catalytic residues of the endonuclease. In some embodiments, the Cas9 molecule is a nickase and produces a single stranded break. See, e.g., Dabrowska et al. Frontiers in Neuroscience (2018) 12(75). It has been shown that one or more mutations in the RuvC and HNH catalytic domains of the enzyme may improve Cas9 efficiency. See, e.g., Sarai et al. Currently Pharma. Biotechnol. (2017) 18(13). In some embodiments, the Cas9 molecule is fused to a second domain, e.g., a domain that modifies DNA or chromatin, e.g., a deaminase or demethylase domain. In some such embodiments, the Cas9 molecule is modified to eliminate its endonuclease activity.

In some embodiments, a Cas nuclease or a Cas/gRNA complex described herein is administered together with a template for homology directed repair (HDR). In some embodiments, a Cas nuclease or a Cas/gRNA complex described herein is administered without a HDR template. In some embodiments, a Cas9 nuclease is used that is modified to enhance specificity of the enzyme (e.g., reduce off-target effects, maintain robust on-target cleavage). In some embodiments, the Cas9 molecule is an enhanced specificity Cas9 variant (e.g., eSPCas9). See, e.g., Slaymaker et al. Science (2016) 351 (6268): 84-88. In some embodiments, the Cas9 molecule is a high fidelity Cas9 variant (e.g., SpCas9-HF1). See, e.g., Kleinstiver et al. Nature (2016) 529: 490-495.

Various Cas nucleases are known in the art and may be obtained from various sources and/or engineered/modified to modulate one or more activities or specificities of the enzymes. PAM sequence preferences and specificities of suitable Cas nucleases, e.g., suitable Cas9 nucleases, such as, for example, SpCas9 and SaCas9 are known in the art. In some embodiments, the Cas nuclease has been engineered/modified to recognize one or more PAM sequence. In some embodiments, the Cas nuclease has been engineered/modified to recognize one or more PAM sequence that is different than the PAM sequence the Cas nuclease recognizes without engineering/modification. In some embodiments, the Cas nuclease has been engineered/modified to reduce off-target activity of the enzyme.

In some embodiments, a Cas nuclease is used that is modified further to alter the specificity of the endonuclease activity (e.g., reduce off-target cleavage, decrease the endonuclease activity or lifetime in cells, increase homology-directed recombination and reduce non-homologous end joining). See, e.g., Komor et al. Cell (2017) 168: 20-36. In some embodiments, a Cas nuclease is used that is modified to alter the PAM recognition or preference of the endonuclease. For example, SpCas9 recognizes the PAM sequence NGG, whereas some variants of SpCas9 comprising one or more modifications (e.g., VQR SpCas9, EQR SpCas9, VRER SpCas9) may recognize variant PAM sequences, e.g., NGA, NGAG, and/or NGCG. For another example, SaCas9 recognizes the PAM sequence NNGRRT, whereas some variants of SaCas9 comprising one or more modifications (e.g., KKH SaCas9) may recognize the PAM sequence NNNRRT. In another example, FnCas9 recognizes the PAM sequence NNG, whereas a variant of the FnCas9 comprises one or more modifications (e.g., RHA FnCas9) may recognize the PAM sequence YG. In another example, the Cas12a nuclease comprising substitution mutations S542R and K607R recognizes the PAM sequence TYCV. In another example, a Cpf1endonuclease comprising substitution mutations S542R, K607R, and N552R recognizes the PAM sequence TATV. See, e.g., Gao et al. Nat. Biotechnol. (2017) 35(8): 789-792.

In some embodiments, a base editor is used to create a genomic modification resulting in a loss of expression of a lineage-specific cell-surface antigen, or expression of a variant of a lineage-specific cell-surface antigen that is not targeted by an immunotherapy. Base editors typically comprise a catalytically inactive or partially inactive Cas nuclease fused to a functional domain, e.g., a deaminase domain. See, e.g., Eid et al. Biochem. J. (2018) 475(11): 1955-1964; Rees et al. Nature Reviews Genetics (2018) 19:770-788. In some embodiments, a catalytically inactive Cas nuclease is referred to as “dead Cas” or “dCas.” In some embodiments, the endonuclease comprises a dCas fused to an adenine base editor (ABE), for example an ABE evolved from the RNA adenine deaminase TadA. In some embodiments, the endonuclease comprises a dCas fused to cytidine deaminase enzyme (e.g., APOBEC deaminase, pmCDA1, activation-induced cytidine deaminase (AID)). In some embodiments, the catalytically inactive Cas molecule has reduced activity and is, e.g., a nickase.

Examples of suitable base editors include, without limitation, BE1, BE2, BE3, HF-BE3, BE4, BE4max, BE4-Gam, YE1-BE3, EE-BE3, YE2-BE3, YEE-CE3, VQR-BE3, VRER-BE3, SaBE3, SaBE4, SaBE4-Gam, Sa(KKH)-BE3, Target-AID, Target-AID-NG, xBE3, eA3A-BE3, BE-PLUS, TAM, CRISPR-X, ABE7.9, ABE7.10, ABE7.10*, xABE, ABESa, VQR-ABE, VRER-ABE, Sa(KKH)-ABE, and CRISPR-SKIP. Additional examples of base editors can be found, for example, in US Publication No. 2018/0312825A1, US Publication No. 2018/0312828A1, and PCT Publication No. WO 2018/165629A1, which are incorporated by reference herein in their entireties.

Some aspects of this disclosure provide guide RNAs that are suitable to target an RNA-guided nuclease, e.g. as provided herein, to a suitable target site in the genome of a cell in order to effect a modification in the genome of the cell that results in reduced expression of a lineage-specific cell-surface antigen, loss of expression of a lineage-specific cell-surface antigen, or expression of a variant form of the lineage-specific cell-surface antigen that is not recognized by an immunotherapeutic agent targeting the lineage-specific cell-surface antigen.

Some exemplary suitable Cas9 gRNA scaffold sequences are provided herein, and additional suitable gRNA scaffold sequences will be apparent to the skilled artisan based on the present disclosure. Such additional suitable scaffold sequences include, without limitation, those recited in Jinek, et al. Science (2012) 337(6096):816-821, Ran, et al. Nature Protocols (2013) 8:2281-2308, PCT Publication No. WO2014/093694, and PCT Publication No. WO2013/176772.

For example, the binding domains of naturally occurring SpCas9 gRNA typically comprise two RNA molecules, the crRNA (partially) and the tracrRNA. Variants of SpCas9 gRNAs that comprise only a single RNA molecule including both crRNA and tracrRNA sequences, covalently bound to each other, e.g., via a tetraloop or via click-chemistry type covalent linkage, have been engineered and are commonly referred to as “single guide RNA” or “sgRNA.” Suitable gRNAs for use with other Cas nucleases, for example, with Cas12a nucleases, typically comprise only a single RNA molecule, as the naturally occurring Cas12a guide RNA comprises a single RNA molecule. A suitable gRNA may thus be unimolecular (having a single RNA molecule), sometimes referred to herein as sgRNAs, or modular (comprising more than one, and typically two, separate RNA molecules).

A gRNA suitable for targeting a target site in a lineage-specific cell-surface antigen may comprise a number of domains. In some embodiments, e.g., in some embodiments where a Cas9 nuclease is used, a unimolecular sgRNA, may comprise, from 5′ to 3′:
a targeting domain corresponding to a target site sequence in the lineage-specific cell-surface antigen encoding gene;
a first complementarity domain;
a linking domain;
a second complementarity domain (which is complementary to the first complementarity domain);
a proximal domain; and
optionally, a tail domain.
Each of these domains is now described in more detail.

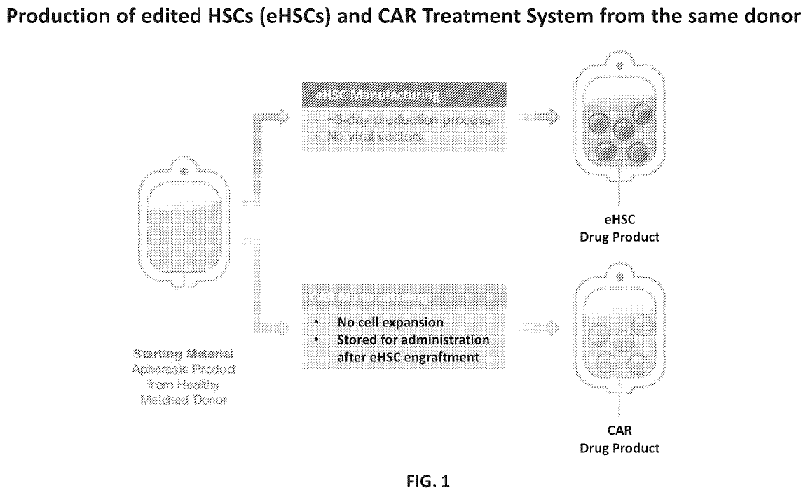

A gRNA as provided herein typically comprises a targeting domain that binds to a target site in the genome of a cell. The target site is typically a double-stranded DNA sequence comprising the PAM sequence and, on the same strand as, and directly adjacent to, the PAM sequence, the target domain. The targeting domain of the gRNA typically comprises an RNA sequence that corresponds to the target domain sequence in that it resembles the sequence of the target domain, sometimes with one or more mismatches, but typically comprises an RNA instead of a DNA sequence. The targeting domain of the gRNA thus base-pairs (in full or partial complementarity) with the sequence of the double-stranded target site that is complementary to the sequence of the target domain, and thus with the strand complementary to the strand that comprises the PAM sequence. It will be understood that the targeting domain of the gRNA typically does not include the PAM sequence. It will further be understood that the location of the PAM may be 5′ or 3′ of the target domain sequence, depending on the nuclease employed. For example, the PAM is typically 3′ of the target domain sequences for Cas9 nucleases, and 5′ of the target domain sequence for Cas12a nucleases. For an illustration of the location of the PAM and the mechanism of gRNA binding a target site, see, e.g., FIG. 1 of Vanegas et al., Fungal Biol Biotechnol. 2019; 6: 6, which is incorporated by reference herein. For additional illustration and description of the mechanism of gRNA targeting an RNA-guided nuclease to a target site, see Fu Y et al, Nat Biotechnol 2014 (doi: 10.1038/nbt.2808) and Sternberg S H et al., Nature 2014 (doi: 10.1038/nature13011), both incorporated herein by reference.

The targeting domain may comprise a nucleotide sequence that corresponds to the sequence of the target domain, i.e., the DNA sequence directly adjacent to the PAM sequence (e.g., 5′ of the PAM sequence for Cas9 nucleases, or 3′ of the PAM sequence for Cas12a nucleases). The targeting domain sequence typically comprises between 17 and 30 nucleotides and corresponds fully with the target domain sequence (i.e., without any mismatch nucleotides), or may comprise one or more, but typically not more than 4, mismatches. As the targeting domain is part of an RNA molecule, the gRNA, it will typically comprise ribonucleotides, while the DNA targeting domain will comprise deoxyribonucleotides.

An exemplary illustration of a Cas9 target site, comprising a 22 nucleotide target domain, and an NGG PAM sequence, as well as of a gRNA comprising a targeting domain that fully corresponds to the target domain (and thus base-pairs with full complementarity with the DNA strand complementary to the strand comprising the target domain and PAM) is provided below:

```
      [ target domain (DNA) ][ PAM ]
5′-N-N-N-N-N-N-N-N-N-N-N-N-N-N-N-N-N-N-N-N-N-N-N-G-G-3′ (DNA)
3′-N-N-N-N-N-N-N-N-N-N-N-N-N-N-N-N-N-N-N-N-N-N-N-C-C-5′ (DNA)
      | | | | | | | | | | | | | | | | | | | | | |
5′-N-N-N-N-N-N-N-N-N-N-N-N-N-N-N-N-N-N-N-N-N-N-[gRNA scaffold]-3′ (RNA)
      [ targeting domain (RNA) ][binding domain]
```

An exemplary illustration of a Cas12a target site, comprising a 22 nucleotide target domain, and a TTN PAM sequence, as well as of a gRNA comprising a targeting domain that fully corresponds to the target domain (and thus base-pairs with full complementarity with the DNA strand complementary to the strand comprising the target domain and PAM) is provided below:

```
                        [ PAM ][ target domain (DNA) ]
                    5′-T-T-N-N-N-N-N-N-N-N-N-N-N-N-N-N-N-N-N-N-N-N-N-N-N-3′ (DNA)
                    3′-A-A-N-N-N-N-N-N-N-N-N-N-N-N-N-N-N-N-N-N-N-N-N-N-N-5′ (DNA)
                                      | | | | | | | | | | | | | | | | | | | | | |
5′-[gRNA scaffold]-N-N-N-N-N-N-N-N-N-N-N-N-N-N-N-N-N-N-N-N-N-N-3′ (RNA)
    [binding domain][ targeting domain (RNA) ]
```

In some embodiments, the Cas12a PAM sequence is 5′-T-T-T-V-3′.

While not wishing to be bound by theory, at least in some embodiments, it is believed that the length and complementarity of the targeting domain with the target sequence contributes to specificity of the interaction of the gRNA/Cas9 molecule complex with a target nucleic acid. In some embodiments, the targeting domain of a gRNA provided herein is 5 to 50 nucleotides in length. In some embodiments, the targeting domain is 15 to 25 nucleotides in length. In some embodiments, the targeting domain is 18 to 22 nucleotides in length. In some embodiments, the targeting domain is 19-21 nucleotides in length. In some embodiments, the targeting domain is 15 nucleotides in length. In some embodiments, the targeting domain is 16 nucleotides in length. In some embodiments, the targeting domain is 17 nucleotides in length. In some embodiments, the targeting domain is 18 nucleotides in length. In some embodiments, the targeting domain is 19 nucleotides in length. In some embodiments, the targeting domain is 20 nucleotides in length. In some embodiments, the targeting domain is 21 nucleotides in length. In some embodiments, the targeting domain is 22 nucleotides in length. In some embodiments, the targeting domain is 23 nucleotides in length. In some embodiments, the targeting domain is 24 nucleotides in length. In some embodiments, the targeting domain is 25 nucleotides in length. In some embodiments, the targeting domain fully corresponds, without mismatch, to a target domain sequence provided herein, or a part thereof. In some embodiments, the targeting domain of a gRNA provided herein comprises 1 mismatch relative to a target domain sequence provided herein. In some embodiments, the targeting domain comprises 2 mismatches relative to the target domain sequence. In some embodiments, the target domain comprises 3 mismatches relative to the target domain sequence.

In some embodiments, a targeting domain comprises a core domain and a secondary targeting domain, e.g., as described in PCT Publication No. WO2015/157070, which is incorporated by reference in its entirety. In some embodiments, the core domain comprises about 8 to about 13 nucleotides from the 3′ end of the targeting domain (e.g., the most 3′ 8 to 13 nucleotides of the targeting domain). In some embodiments, the secondary domain is positioned 5′ to the core domain. In some embodiments, the core domain corresponds fully with the target domain sequence, or a part thereof. In other embodiments, the core domain may comprise one or more nucleotides that are mismatched with the corresponding nucleotide of the target domain sequence.

In some embodiments, e.g., in some embodiments where a Cas9 gRNA is provided, the gRNA comprises a first complementarity domain and a second complementarity domain, wherein the first complementarity domain is complementary with the second complementarity domain, and, at least in some embodiments, has sufficient complementarity to the second complementarity domain to form a duplexed region under at least some physiological conditions. In some embodiments, the first complementarity domain is 5 to 30 nucleotides in length. In some embodiments, the first complementarity domain comprises 3 subdomains, which, in the 5′ to 3′ direction are: a 5′ subdomain, a central subdomain, and a 3′ subdomain. In some embodiments, the 5′ subdomain is 4 to 9, e.g., 4, 5, 6, 7, 8 or 9 nucleotides in length. In some embodiments, the central subdomain is 1, 2, or 3, e.g., 1, nucleotide in length. In some embodiments, the 3′ subdomain is 3 to 25, e.g., 4 to 22, 4 to 18, or 4 to 10, or 3, 4, 5, 6, 7, 8, 9, 10, 11, 12, 13, 14, 15, 16, 17, 18, 19, 20, 21, 22, 23, 24, or 25 nucleotides in length. The first complementarity domain can share homology with, or be derived from, a naturally occurring first complementarity domain. In an embodiment, it has at least 50% homology with a S. pyogenes, S. aureus, or S. thermophilus, first complementarity domain.

The sequence and placement of the above-mentioned domains are described in more detail in PCT Publication No. WO2015/157070, which is herein incorporated by reference in its entirety, including p. 88-112 therein.

A linking domain may serve to link the first complementarity domain with the second complementarity domain of a unimolecular gRNA. The linking domain can link the first and second complementarity domains covalently or non-covalently. In some embodiments, the linkage is covalent. In some embodiments, the linking domain is, or comprises, a covalent bond interposed between the first complementarity domain and the second complementarity domain. In some embodiments, the linking domain comprises one or more, e.g., 2, 3, 4, 5, 6, 7, 8, 9, or 10 nucleotides. In some embodiments, the linking domain comprises at least one non-nucleotide bond, e.g., as disclosed in PCT Publication No. WO2018/126176, the entire contents of which are incorporated herein by reference. In some embodiments, the second complementarity domain is complementary, at least in part, with the first complementarity domain, and in an embodiment, has sufficient complementarity to the second complementarity domain to form a duplexed region under at least some physiological conditions. In some embodiments, the second complementarity domain can include a sequence that lacks complementarity with the first complementarity domain, e.g., a sequence that loops out from the duplexed region. In some embodiments, the second complementarity domain is 5 to 27 nucleotides in length. In some embodiments, the second complementarity domain is longer than the first complementarity region. In an embodiment, the complementary domain is 5, 6, 7, 8, 9, 10, 11, 12, 13, 14, 15, 16, 17, 18, 19, 20, 21, 22, 23, 24, 25, 26, or 27 nucleotides in length. In some embodiments, the second complementarity domain comprises 3 subdomains, which, in the 5′ to 3′ direction are: a 5′ subdomain, a central subdomain, and a 3′ subdomain. In some embodiments, the 5′ subdomain is 3 to 25, e.g., 4 to 22, 4 to 18, or 4 to 10, or 3, 4, 5, 6, 7, 8, 9, 10, 11, 12, 13, 14, 15, 16, 17, 18, 19, 20, 21, 22, 23, 24, 25, 26, or 27 nucleotides in length. In some embodiments, the central subdomain is 1, 2, 3, 4 or 5, e.g., 3, nucleotides in length. In some embodiments, the 3′ subdomain is 4 to 9, e.g., 4, 5, 6, 7, 8 or 9 nucleotides in length. In some embodiments, the 5′ subdomain and the 3′ subdomain of the first complementarity domain, are respectively, complementary, e.g., fully complementary, with the 3′ subdomain and the 5′ subdomain of the second complementarity domain.

In some embodiments, the proximal domain is 5 to 20 nucleotides in length. In some embodiments, the proximal domain can share homology with or be derived from a naturally occurring proximal domain. In an embodiment, it has at least 50% homology with a proximal domain from S. pyogenes, S. aureus, or S. thermophilus.

A broad spectrum of tail domains are suitable for use in gRNAs. In some embodiments, the tail domain is 0 (absent), 1, 2, 3, 4, 5, 6, 7, 8, 9, or 10 nucleotides in length. In some embodiments, the tail domain nucleotides are from or share homology with a sequence from the 5′ end of a naturally occurring tail domain. In some embodiments, the tail domain includes sequences that are complementary to each other and which, under at least some physiological conditions, form a duplexed region. In some embodiments, the tail domain is absent or is 1 to 50 nucleotides in length. In some embodiments, the tail domain can share homology with or be derived from a naturally occurring proximal tail domain. In some embodiments, the tail domain has at least 50% homology/identity with a tail domain from S. pyogenes, S. aureus or S. thermophilus. In some embodiments, the tail domain includes nucleotides at the 3′ end that are related to the method of in vitro or in vivo transcription.

In some embodiments, a gRNA provided herein comprises:
a first strand comprising, e.g., from 5′ to 3′:
a targeting domain (which corresponds to a target domain in the CD30 gene); and
a first complementarity domain; and
a second strand, comprising, e.g., from 5′ to 3′:
optionally, a 5′ extension domain;
a second complementarity domain;
a proximal domain; and
optionally, a tail domain.

In some embodiments, any of the gRNAs provided herein comprise one or more nucleotides that are chemically modified. Chemical modifications of gRNAs have previously been described, and suitable chemical modifications include any modifications that are beneficial for gRNA function and do not measurably increase any undesired characteristics, e.g., off-target effects, of a given gRNA. Suitable chemical modifications include, for example, those that make a gRNA less susceptible to endo- or exonuclease catalytic activity, and include, without limitation, phosphorothioate backbone modifications, 2′-O—Me-modifications (e.g., at one or both of the 3′ and 5′ termini), 2′F-modifications, replacement of the ribose sugar with the bicyclic nucleotide-cEt, 3′thioPACE (MSP) modifications, or any combination thereof. Additional suitable gRNA modifications will be apparent to the skilled artisan based on this disclosure, and such suitable gRNA modifications include, without limitation, those described, e.g., in Randar et al. PNAS (2015) 112 (51) E7110-E7117 and Hendel et al., Nat Biotechnol. (2015); 33(9): 985-989, each of which is incorporated herein by reference in its entirety.

For example, a gRNA provided herein may comprise one or more 2′-O modified nucleotide, e.g., a 2′-O-methyl nucleotide. In some embodiments, the gRNA comprises a 2′O-modified nucleotide, e.g., 2′-O-methyl nucleotide at the 5′ end of the gRNA. In some embodiments, the gRNA comprises a 2′-O modified nucleotide, e.g., 2′-O-methyl nucleotide at the 3′ end of the gRNA. In some embodiments, the gRNA comprises a 2′-O-modified nucleotide, e.g., a 2′-O-methyl nucleotide at both the 5′ and 3′ ends of the gRNA. In some embodiments, the gRNA is 2′-O-modified, e.g. 2′-O-methyl-modified at the nucleotide at the 5′ end of the gRNA, the second nucleotide from the 5′ end of the gRNA, and the third nucleotide from the 5′ end of the gRNA. In some embodiments, the gRNA is 2′-O-modified, e.g. 2′-O-methyl-modified at the nucleotide at the 3′ end of the gRNA, the second nucleotide from the 3′ end of the gRNA, and the third nucleotide from the 3′ end of the gRNA. In some embodiments, the gRNA is 2′-O-modified, e.g. 2′-O-methyl-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified, e.g. 2′-O-methyl-modified at the second nucleotide from the 3′ end of the gRNA, the third nucleotide from the 3′ end of the gRNA, and at the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified, e.g. 2′-O-methyl-modified, at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA. In some embodiments, the 2′-O-methyl nucleotide comprises a phosphate linkage to an adjacent nucleotide. In some embodiments, the 2′-O-methyl nucleotide comprises a phosphorothioate linkage to an adjacent nucleotide. In some embodiments, the 2′-O-methyl nucleotide comprises a thioPACE linkage to an adjacent nucleotide.

In some embodiments, a gRNA provided herein may comprise one or more 2′-O-modified and 3′phosphorous-modified nucleotide, e.g., a 2′-O-methyl 3′phosphorothioate nucleotide. In some embodiments, the gRNA comprises a 2′-O-modified and 3′phosphorous-modified, e.g., 2′-O-methyl 3′phosphorothioate nucleotide at the 5′ end of the gRNA. In some embodiments, the gRNA comprises a 2′-O-modified and 3′phosphorous-modified, e.g., 2′-O-methyl 3′phosphorothioate nucleotide at the 3′ end of the gRNA. In some embodiments, the gRNA comprises a 2′-O-modified and 3′phosphorous-modified, e.g., 2′-O-methyl 3′phosphorothioate nucleotide at the 5′ and 3′ ends of the gRNA. In some embodiments, the gRNA comprises a backbone in which one or more non-bridging oxygen atoms has been replaced with a sulfur atom. In some embodiments, the gRNA is 2′-O-modified and 3′phosphorous-modified, e.g. 2′-O-methyl 3′phosphorothioate-modified at the nucleotide at the 5′ end of the gRNA, the second nucleotide from the 5′ end of the gRNA, and the third nucleotide from the 5′ end of the gRNA. In some embodiments, the gRNA is 2′-O-modified and 3′phosphorous-modified, e.g. 2′-O-methyl 3′phosphorothioate-modified at the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′phosphorothioate-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′phosphorothioate-modified at the second nucleotide from the 3′ end of the gRNA, the third nucleotide from the 3′ end of the gRNA, and the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified and 3′phosphorous-modified, e.g. 2′-O-methyl 3′phosphorothioate-modified at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA.

In some embodiments, a gRNA provided herein may comprise one or more 2′-O-modified and 3′-phosphorous-modified, e.g., 2′-O-methyl 3′thioPACE nucleotide. In some embodiments, the gRNA comprises a 2′-O-modified and 3′phosphorous-modified, e.g., 2′-O-methyl 3′thioPACE nucleotide at the 5′ end of the gRNA. In some embodiments, the gRNA comprises a 2′-O-modified and 3′phosphorous-modified, e.g., 2′-O-methyl 3′thioPACE nucleotide at the 3′ end of the gRNA. In some embodiments, the gRNA comprises a 2′-O-modified and 3′phosphorous-modified, e.g., 2′-O-methyl 3′thioPACE nucleotide at the 5′ and 3′ ends of the gRNA. In some embodiments, the gRNA comprises a backbone in which one or more non-bridging oxygen atoms have been replaced with a sulfur atom and one or more non-bridging oxygen atoms have been replaced with an acetate group. In some embodiments, the gRNA is 2′-O-modified and 3′phosphorous-modified, e.g. 2′-O-methyl 3′ thioPACE-modified at the nucleotide at the 5′ end of the gRNA, the second nucleotide from the 5′ end of the gRNA, and the third nucleotide from the 5′ end of the gRNA. In some embodiments, the gRNA is 2′-O-modified and 3′phosphorous-modified, e.g. 2′-O-methyl 3′thioPACE-modified at the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′thioPACE-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′thioPACE-modified at the second nucleotide from the 3′ end of the gRNA, the third nucleotide from the 3′ end of the gRNA, and the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified and 3′phosphorous-modified, e.g. 2′-O-methyl 3′thioPACE-modified at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA.

In some embodiments, a gRNA provided herein comprises a chemically modified backbone. In some embodiments, the gRNA comprises a phosphorothioate linkage. In some embodiments, one or more non-bridging oxygen atoms have been replaced with a sulfur atom. In some embodiments, the nucleotide at the 5′ end of the gRNA, the second nucleotide from the 5′ end of the gRNA, and the third nucleotide from the 5′ end of the gRNA each comprise a phosphorothioate linkage. In some embodiments, the nucleotide at the 3′ end of the gRNA, the second nucleotide from the 3′ end of the gRNA, and the third nucleotide from the 3′ end of the gRNA each comprise a phosphorothioate linkage. In some embodiments, the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA each comprise a phosphorothioate linkage. In some embodiments, the second nucleotide from the 3′ end of the gRNA, the third nucleotide from the 3′ end of the gRNA, and at the fourth nucleotide from the 3′ end of the gRNA each comprise a phosphorothioate linkage. In some embodiments, the nucleotide at the 5′ end of the gRNA, the second nucleotide from the 5′ end of the gRNA, the third nucleotide from the 5′ end, the second nucleotide from the 3′ end of the gRNA, the third nucleotide from the 3′ end of the gRNA, and the fourth nucleotide from the 3′ end of the gRNA each comprise a phosphorothioate linkage.

In some embodiments, a gRNA provided herein comprises a thioPACE linkage. In some embodiments, the gRNA comprises a backbone in which one or more non-bridging oxygen atoms have been replaced with a sulfur atom and one or more non-bridging oxygen atoms have been replaced with an acetate group. In some embodiments, the nucleotide at the 5′ end of the gRNA, the second nucleotide from the 5′ end of the gRNA, and the third nucleotide from the 5′ end of the gRNA each comprise a thioPACE linkage. In some embodiments, the nucleotide at the 3′ end of the gRNA, the second nucleotide from the 3′ end of the gRNA, and the third nucleotide from the 3′ end of the gRNA each comprise a thioPACE linkage. In some embodiments, the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA each comprise a thioPACE linkage. In some embodiments, the second nucleotide from the 3′ end of the gRNA, the third nucleotide from the 3′ end of the gRNA, and at the fourth nucleotide from the 3′ end of the gRNA each comprise a thioPACE linkage. In some embodiments, the nucleotide at the 5′ end of the gRNA, the second nucleotide from the 5′ end of the gRNA, the third nucleotide from the 5′ end, the second nucleotide from the 3′ end of the gRNA, the third nucleotide from the 3′ end of the gRNA, and the fourth nucleotide from the 3′ end of the gRNA each comprise a thioPACE linkage.

In some embodiments, a gRNA described herein comprises one or more 2′-O-methyl-3′-phosphorothioate nucleotides, e.g., at least 1, 2, 3, 4, 5, or 6 2′-O-methyl-3′-phosphorothioate nucleotides. In some embodiments, a gRNA described herein comprises modified nucleotides (e.g., 2′-O-methyl-3′-phosphorothioate nucleotides) at one or more of the three terminal positions and the 5′ end and/or at one or more of the three terminal positions and the 3′ end. In some embodiments, the gRNA may comprise one or more modified nucleotides, e.g., as described in PCT Publication Nos. WO2017/214460, WO2016/089433, and WO2016/164356, which are incorporated by reference their entirety.

### Methods of Use

Some aspects of this disclosure provide methods, comprising administering a plurality of cells provided herein to a subject in need thereof. For example, some aspects of this disclosure provide methods comprising administering a plurality, and preferably an effective number, of cells provided herein, e.g., a genetically engineered cell comprising a heterologous nucleic acid encoding a transgene (e.g., a CAR) targeting a lineage-specific cell-surface antigen associated with a hyperproliferative/neoplastic/malignant disease (wherein the cell is a mobilized lymphocyte cell), to a subject in need thereof. In some embodiments, the genetically engineered lymphocytes expressing the CAR are T lymphocytes. In some embodiments, the genetically engineered lymphocytes expressing the CAR are B lymphocytes. In some embodiments, the genetically engineered lymphocytes expressing the CAR are NK cells.

In some embodiments, the subject is also administered a plurality, and preferably an effective number, of genetically engineered hematopoietic stem cells, wherein the genetically engineered hematopoietic stem cells are characterized by: reduced expression or a lack of expression of the lineage-specific cell surface antigen, or expression of a variant form of the lineage-specific cell-surface antigen that is not recognized or recognized at a reduced level by the CAR. In some embodiments, the cells are HSCs or HPCs.

In some embodiments, the methods further comprise monitoring at least one symptom of the hyperproliferative disease.

Some aspects of this disclosure provide methods of administering a cell provided herein, e.g., a genetically engineered lymphocyte expressing a CAR targeting a lineage-specific cell-surface antigen to a subject having a hyperproliferative disease, e.g., a hematopoietic malignancy such as a myeloid malignancy or lymphoid malignancy. In some embodiments, the subject has the hyperproliferative disease or has been diagnosed with the hyperproliferative disease. In some embodiments, the subject has the hyperproliferative disease or has been diagnosed with the hyperproliferative disease, but the disease not yet entered the malignant state, such as a pre-malignant stage of the disease.

In some embodiments, administration of the cells to the subject ameliorates a sign or symptom associated with the hyperproliferative disease which may include, e.g., reducing the number of neoplastic or malignant cells, reducing tumor burden, including inhibiting growth of a tumor, slowing the growth rate of a tumor, reducing the size of a tumor, reducing the number of tumors, eliminating a tumor, or reducing or ameliorating a symptom associated with the neoplastic disease or disorder, e.g., fatigue, pain, weight loss, and other clinical measures.

In some embodiments, the subject is a mammal. In some embodiments, the subject is a human subject.

In some embodiments, the subject has or has been diagnosed with a hyperproliferative disease. In some embodiments, the hyperproliferative disease is a hematopoietic malignancy. In some embodiments, the hematopoietic malignancy is a myeloid malignancy. In some embodiments, the hematopoietic malignancy is a lymphoid malignancy. In some embodiments, the hematopoietic malignancy is Hodgkin's lymphoma, non-Hodgkin's lymphoma, leukemia, or multiple myeloma. In some embodiments, the leukemia is acute myeloid leukemia, myelodysplastic syndrome, acute lymphoid leukemia, chronic myelogenous leukemia, acute lymphoblastic leukemia or chronic lymphoblastic leukemia, or chronic lymphoid leukemia. In some embodiments, the cancer is acute myeloid leukemia (AML). In some embodiments, the neoplastic disease is myelodysplastic syndrome.

In some embodiments, the hyperproliferative disease is a sarcoma. In some embodiments, the hyperproliferative disease is a melanoma. In some embodiments, the hyperproliferative disease is a brain or spinal cord tumor. In some embodiments, the hyperproliferative disease is a germ cell tumor. In some embodiments, the hyperproliferative disease is a neuroendocrine tumor. In some embodiments, the hyperproliferative disease is a carcinoid tumor. In some embodiments, the hyperproliferative disease is a cancer of a hematopoietic lineage. In some embodiments, the hyperproliferative disease is metastatic cancer.

Depending on the CAR employed and cells provided herein, various diseases or malignancies can be treated, including, but not limited to bone cancer, intestinal cancer, liver cancer, skin cancer, cancer of the head or neck, melanoma (cutaneous or intraocular malignant melanoma), renal cancer (for example, clear cell carcinoma), throat cancer, prostate cancer (for example, hormone refractory prostate adenocarcinoma), blood cancers (for example, leukemias, lymphomas, and myelomas), uterine cancer, rectal cancer, cancer of the anal region, bladder cancer, brain cancer, stomach cancer, testicular cancer, carcinoma of the fallopian tubes, carcinoma of the endometrium, carcinoma of the cervix, carcinoma of the vagina, carcinoma of the vulva, leukemias (for example, acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease, Waldenstrom's macroglobulinemia), cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, lymphocytic lymphoma, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, heavy chain disease, and solid tumors such as sarcomas and carcinomas, for example, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, hepatoma, bile duct carcinoma, choriocarcinoma, seminoma, embryonal carcinoma, Wilm's tumor, cervical cancer, uterine cancer, testicular cancer, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, retinoblastoma, malignant pleural disease, mesothelioma, lung cancer (for example, non-small cell lung cancer), pancreatic cancer, ovarian cancer, breast cancer (for example, metastatic breast cancer, metastatic triple-negative breast cancer), colon cancer, pleural tumor, glioblastoma, esophageal cancer, gastric cancer, and synovial sarcoma. Solid tumors can be primary tumors or tumors in a metastatic state.

In some embodiments, genetically engineered cells as provided herein, e.g., genetically engineered lymphocytes expressing CARs, genetically engineered hematopoietic stem cells, are administered to a subject in need thereof at a dose of about 10⁴ to about 10¹⁰ cells/kg of body weight of the subject, for example, about 10⁵ to about 10⁹, about 10⁵ to about 10⁸, about 10⁵ to about 10⁷, or about 10⁵ to 10⁶. In general, in the case of systemic administration, a higher dose is used than in regional administration. The dose(s) of any of the cells described herein can also be adjusted to account for whether a single dose is being administered or whether multiple doses are being administered. The precise determination of what would be considered an effective dose can be based on factors individual to each subject, including their size, age, sex, weight, and condition of the particular subject, as described above. Dosages can be readily determined by those skilled in the art based on the disclosure herein and knowledge in the art.

The genetically engineered cells provided herein can be administered by any methods known in the art, including, but not limited to, pleural administration, intravenous administration, subcutaneous administration, intranodal administration, intratumoral administration, intrathecal administration, intrapleural administration, intraperitoneal administration, intracranial administration, and direct administration to the thymus.

### EXAMPLES

### Example 1

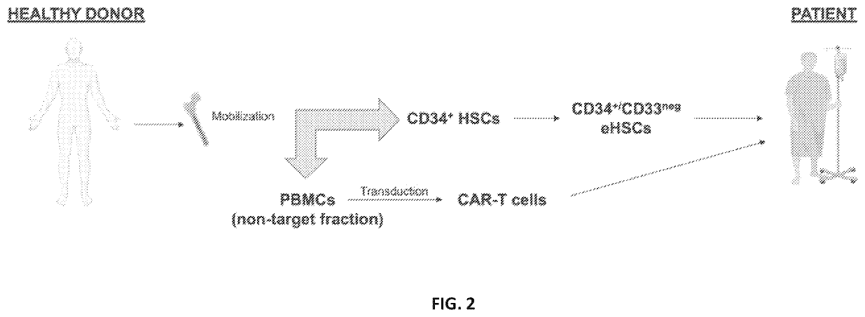

As shown in the exemplary schematic shown in FIGS. 1 and 2, edited hematopoietic stem cells and cells that are genetically engineered to express a chimeric antigen receptor (CAR) may be produced from a single starting material.

Briefly, a donor (e.g., a healthy, HLA matched donor) is administered a mobilization agent, such as any of those described herein, that promotes mobilization of cells typically located in the bone marrow of the subject. An apheresis product is obtained from a donor subject that contains a heterogenous population of mobilized cells, including hematopoietic stem cells and PMBCs. The CD34+ cells (HSCs, which may be referred to as the “target cells”) are isolated from the starting material and used to produce edited hematopoietic stem cell (eHSC) drug product. For example, the hematopoietic stem cells may be genetically engineered to have reduced expression or lack expression of a lineage-specific cell-surface antigen, such as CD33 (CD34+/CD33^{neg} eHSCs).

The remaining population of cells, which typically may be discarded (referred to as the “non-target fraction”), is genetically engineered by transducing a heterologous nucleic acid encoding a CAR to produce a CAR drug product, e.g, CAR-T cells. The CAR drug product cells may be stored for later administration to the patient following engraftment of the eHSC.

### Example 2: G-CSF/Plerixafor Dual-Mobilized Donor Derived CD33 CAR-T cells as Potent and Effective AML Therapy in Pre-Clinical Models

There are currently no acute myeloid leukemia (AML) specific antigens. Genetic ablation of CD33 using CRISPR/Cas9 engineering of the hematopoietic stem cells (HSCs) for transplant represents a synthetic biology approach to generating a leukemia-specific antigen in the transplant recipient. Transplant of CD33KO HSCs allows for CD33-targeted immunotherapeutic (e.g., anti-CD33 CAR-T cell mediated) killing of AML cells while sparing edited CD33KO HSCs, and thus, e.g., continued myeloid development and function sustained by these CD33Ko HSCs, thereby mitigating the on-target, off-cancer toxicities of CD33-targeted immunotherapeutics.

Mobilized leukapheresis represents an attractive starting material for the generation of both the edited, CD33 null (CD33⁻ or CD33KO) HSCs for the transplant and the complementary immunotherapeutic agent, e.g., a CD33 CAR-T cell product. The impact of dual mobilization with Granulocyte-Colony Stimulating Factor (G-CSF) and Plerixafor (Mozobil) on immune cell composition, T cell phenotype, and the functionality of these T cells to control AML tumor growth upon chimeric antigen receptor (CAR) transduction is described below.

Mobilized (“mob”) PBMCs were collected from healthy donors injected with G-CSF (10 μg/kg/day, 5 consecutive days) and Plerixafor (240 μg/kg, on day 4 and 5). Non-mobilized (“non-mob,” also sometimes referred to herein and in the drawings as “steady state,” or “ss”) PBMCs, collected from the same donors, were used as controls. Cells were analyzed by flow cytometry for immunophenotyping and T cell characterization including differentiation and bone marrow homing markers, as well as responses to T cell activation with anti-CD3 (OKT3) and IL-2.

To evaluate cell types that make up the mobilized cell population, global immune phenotyping was performed using flow cytometric analysis. The mobilized cell population was found to contain populations of T cells, monocytes, NK cells, and B cells. See, FIGS. 3A-3D. As shown in FIG. 3E, the relative abundance of the particular cell types present in the mobilized cell population differed from the abundances of cell types in steady state cell populations. For example, the relative abundance of CD3+ cells (T cells) was reduced in the mobilized cell population as compared to steady stage, whereas the relative abundance of B cells was increased in the mobilized cell population as compared to steady state.

The phenotype of T cells in the mobilized cell population was further assessed. As shown in FIGS. 3F-3H, naïve, effector, and memory T cells were present in the mobilized cell population, however the relative abundance of naïve T cells was increased in the mobilized cell population as compared to the stead state. In addition, the relative abundances of effector and memory T cells were reduced in the mobilized cell population as compared to the stead state.

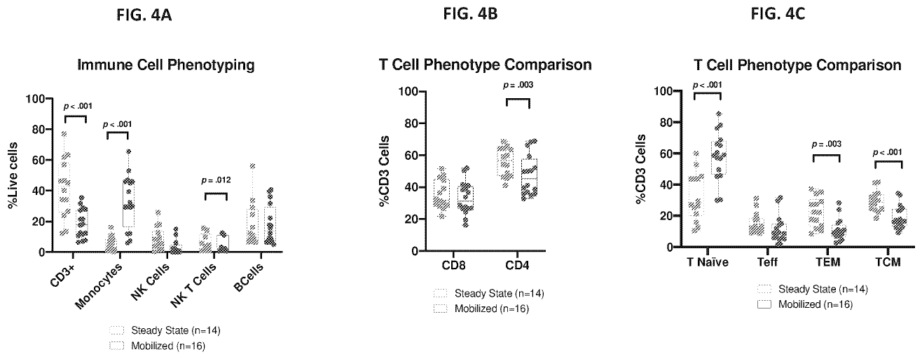

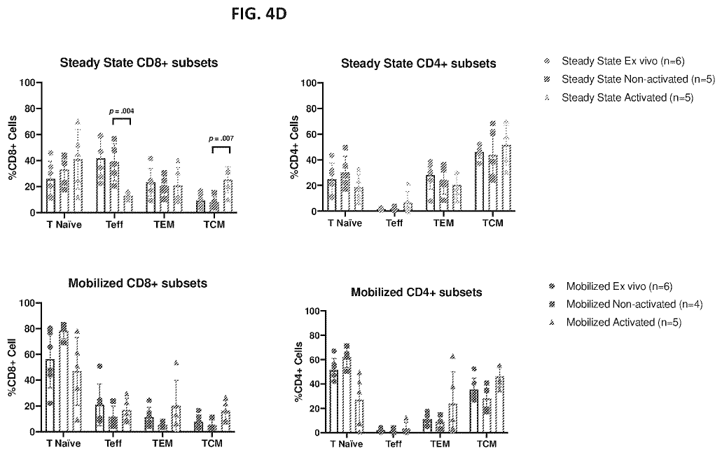

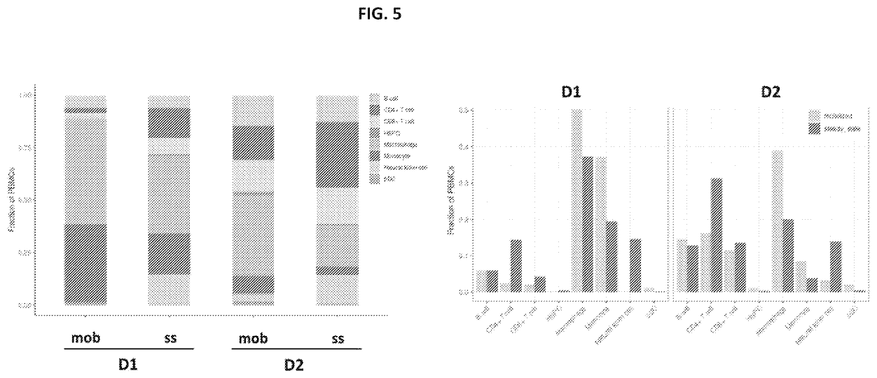

Immune cell and T-cell phenotyping as described above was performed on non-mobilized and mobilized PBMC populations from a plurality of donors. See FIGS. 4A-4C. Different T-cell subsets (CD8⁺ or CD4⁺) obtained from non-mobilized and mobilized PBMC populations were also analyzed, as shown in FIG. 4D. Ex vivo immunophenotyping of PBMC from a total of 30 healthy donors showed that mobilization decreases the overall numbers of CD3⁺ T cells but increases the number of naïve T cells (CD45RA⁺/CCR7⁺), at the expense of T effector-memory (CD45RA⁻/CCR7⁻) and central-memory (CD45RA⁻/CCR7⁺) populations.

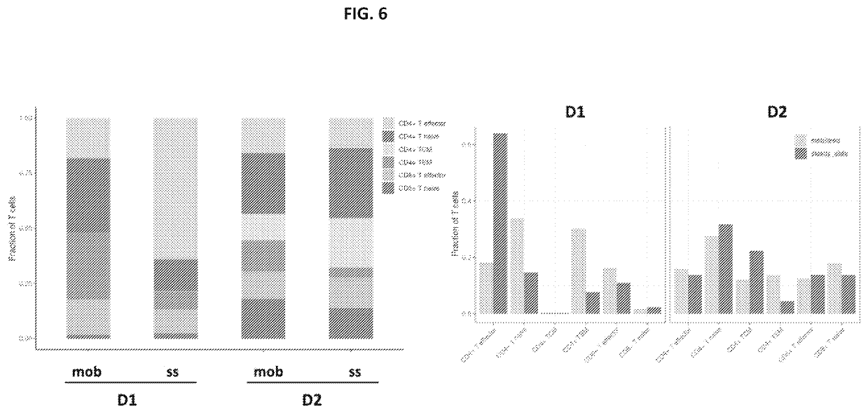

Non-mobilized and mobilized PBMC populations were also analyzed by single-cell next generation sequencing (CITEseq) using 127 immune cell phenotypic markers in combination with extensive transcriptome and T cell receptor repertoire analysis. CITEseq results for two donors (D1 and D2) are shown in FIG. 5 (PBMC population analysis) and FIG. 6 (T-cell subset analysis). Single cell sequencing analyses confirmed mobilization-induced increases in T naïve cells as well as shifts in monocytes/macrophages, CD4⁺ T cells and NK cells percentages.

In addition, it was observed that bone marrow homing factors (e.g.: sialyl-Lewis^{X}, CXCR4) were somewhat decreased in mobilized compared to non-mobilized T cell samples.

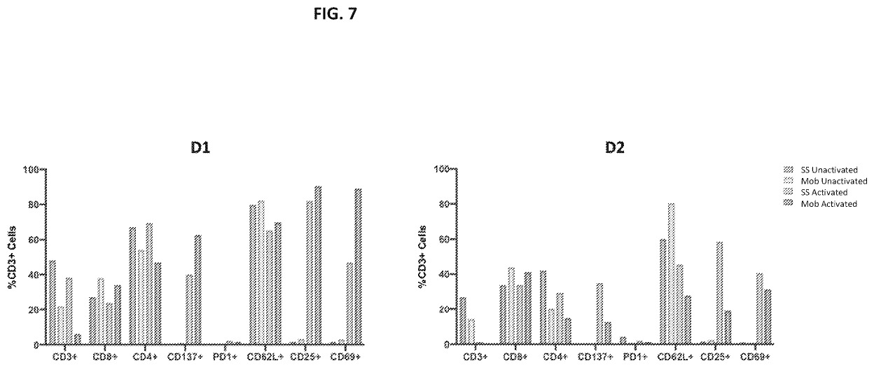

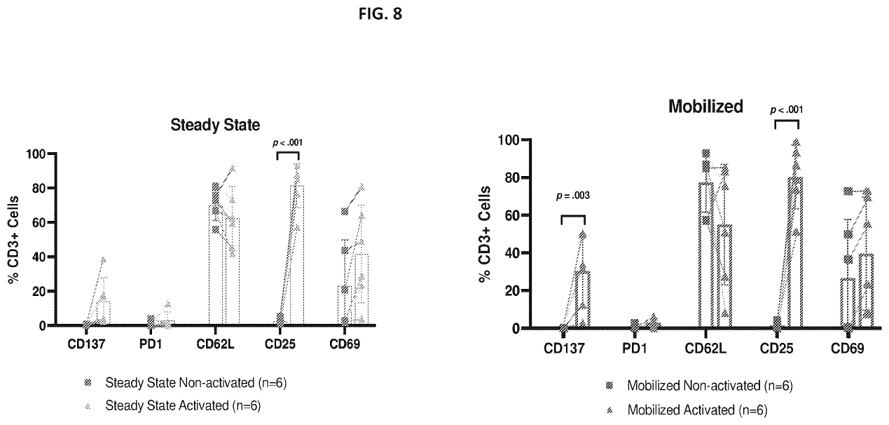

T cell activation (using a standard anti-CD3 and IL-2 protocol) led to similar increases in CD25, CD69, and CD137 expression, and a decrease in CD62L expression, across non-mobilized and mobilized PBMC populations. FIG. 7 illustrates changes in protein expression for non-mobilized and mobilized PBMC populations obtained from two donors (D1 and D2), and FIG. 8 illustrates activation-induced changes in marker expression for a larger donor cohort. Higher numbers of monocytes (CD14⁺) were detected in mobilized compared to non-mobilized donor samples, but disappeared after culture under T cell activation conditions.

Lentiviral transduction of anti-CD33 CAR constructs enabled functional comparisons of mob- and non-mob-CAR T-cells in AML cell co-cultures as well as AML mouse models.

Lentiviral constructs encoded an anti-CD33 CAR comprising an M195 (anti-CD33) binder, a CD28 hinge and transmembrane domain, and a CD3 zeta signaling domain, and CAR-T cells were obtained from mobilized and non-mobilized PBMC fractions by standard lentiviral transduction protocols (see, e.g., PCT/US2020/022309 for reference). The sequence of the CAR is provided below:

(SEQ ID NO: 1)
MALPVTALLLPLALLLHAARPQVQLVQSGAEVKKPGSSVKVSCKASGYTF
TDYNMHWVRQAPGQGLEWIGYIYPYNGGTGYNQKFKSKATITADESTNTA
YMELSSLRSEDTAVYYCARGRPAMDYWGQGTLVTVSSGGGGSGGGGSGGG
GSDIQMTQSPSSLSASVGDRVTITCRASESVDNYGISFMNWFQQKPGKAP
KLLIYAASNQGSGVPSRFSGSGSGTDFTLTISSLQPDDFATYYCQQSKEV
PWTFGQGTKVEIKSGAAAIEVMYPPPYLDNEKSNGTIIHVKGKHLCPSPL
FPGPSKPFWVLVVVGGVLACYSLLVTVAFIIFWVRSKRSRLLHSDYMNMT
PRRPGPTRKHYQPYAPPRDFAAYRSRVKFSRSADAPAYQQGQNQLYNELN
LGRREEYDVLDKRRGRDPEMGGKPRRKNPQEGLYNELQKDKMAEAYSEIG
MKGERRRGKGHDGLYQGLSTATKDTYDALHMQALPPRGS

Functional in vitro cytotoxic assays demonstrated that mob-CD33-CAR T-cells are as effective as non-mob-CD33-CAR T-cells in killing CD33⁺ AML cells, with reduced ‘bystander’ activation of non-transduced T cells (FIGS. 9-11). Non-mobilized PBMC populations yielded a CAR-expression frequency of about 28%, while mobilized PBMC populations yielded about 27% CAR-expressing cells. Target cells included CD33-expressing MOLM13 cells (wt MOLM), CD33 null MOLM13 cells (CD33KO MOLM), and Jurkat cells (no CD33 expression). CAR-T cells and target cells were co-cultured for 24 hours. Control conditions included cells without stimulation (negative control) and cells with standard PMA/Ionomycin stimulation (PMA/I, positive control). In some instances, MYLOTARG™ was used as an additional positive control. The assays were performed at least in duplicate.

FIG. 9 demonstrates that the cytotoxic potential of mobilized-PBMC-derived CAR-T cells equals that of non-mobilized-PBMC-derived CAR-T cells.

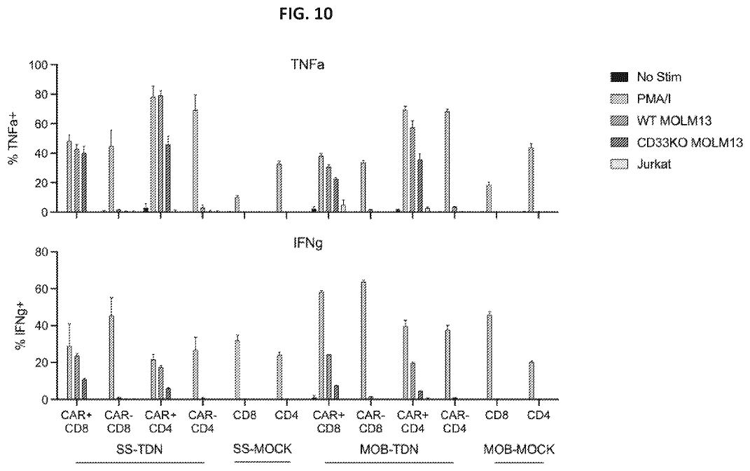

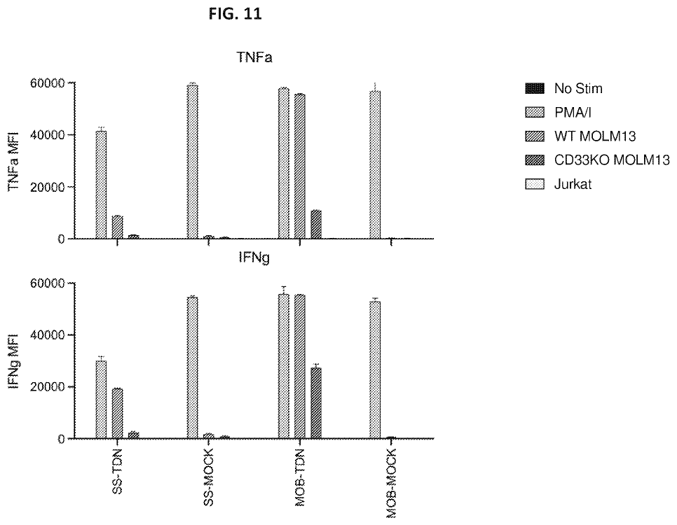

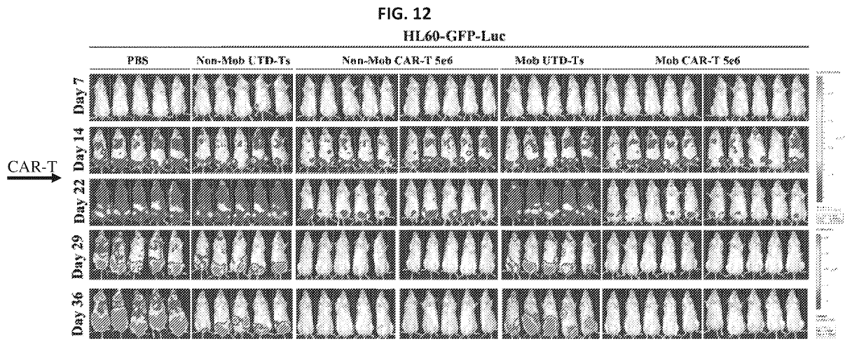

FIG. 10 demonstrates that intracellular activation markers are upregulated in a similar fashion in mobilized and non-mobilized PBMC-derived CAR-T cells, with minimal “bystander” activation, and that both CD8+ and CD8− T cells are activated to produce IFNγ and TNFα in a CAR and antigen-dependent manner.

FIG. 11 shows LUMINEX™ analyses of supernatant cytokines, confirming the results of equivalent cytotoxic potential of non-mobilized and mobilized PBMC-derived CAR-T cells.

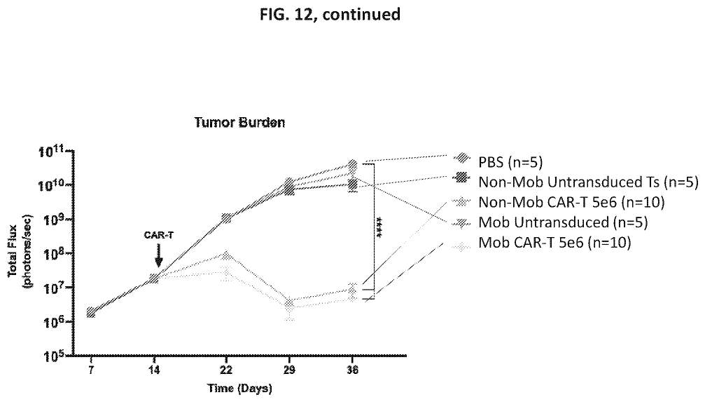

Assessment of the cytotoxic potential of non-mobilized and mobilized PBMC-derived CAR-T cells in an in vivo AML mouse model are shown in FIG. 12, demonstrating that mobilized-CD33-CAR T-cells are equally effective in clearing CD33⁺ tumors as non-mobilized-CD33-CAR T-cells.

The data shown herein demonstrate phenotypical ex vivo differences between mob and non-mob PBMCs, which largely disappeared upon activation, indicating similar responses to T cell-specific stimulation. These findings are corroborated by similar in vitro cytotoxicity profiles of non-/mob-CAR T-cells. Non-transduced T cells in the mob-CAR T-cell population showed limited ‘bystander’ activation, indicating a potentially favorable clinical toxicity profile. Additional in vivo assessment of mob-CAR T-cell function shows effective tumor clearance, which supports further efforts towards their clinical use in combination with engineered HSCs for the treatment of AML patients.

### REFERENCES

All publications, patents, patent applications, publication, and database entries (e.g., sequence database entries) mentioned herein, e.g., in the Background, Summary, Detailed Description, Examples, and/or References sections, are hereby incorporated by reference in their entirety as if each individual publication, patent, patent application, publication, and database entry was specifically and individually incorporated herein by reference. In case of conflict, the present application, including any definitions herein, will control.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the invention encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods described herein, can be excluded from any one or more claims. For purposes of brevity, all of the embodiments in which one or more elements, features, purposes, or aspects is excluded are not set forth explicitly herein.

We claim:

1. A genetically engineered cell, comprising
a heterologous nucleic acid encoding a chimeric antigen receptor (CAR) targeting a lineage-specific cell-surface antigen associated with a hyperproliferative disease,
wherein the cell is a mobilized lymphocyte cell, or a descendant thereof.

2. The genetically engineered cell of claim 1, wherein the cell is a T lymphocyte.

3. The genetically engineered cell of claim 2, wherein the T lymphocyte expresses CD3, CD4, and/or CD8.

4. The genetically engineered cell of claim 2 or 3, wherein the T lymphocyte expresses CD4 and CD8.

5. The genetically engineered cell of any one of claims 2-4, wherein the T lymphocyte expresses PD1.

6. The genetically engineered cell of any one of claims 1-5, wherein the T lymphocyte is an alpha/beta T lymphocyte.

7. The genetically engineered cell of any one of claims 1-5, wherein the T lymphocyte is a gamma/delta T lymphocyte.

8. The genetically engineered cell of any one of claims 1-7, wherein the T lymphocyte is a naïve T lymphocyte.

9. The genetically engineered cell of any one of claims 1-7, wherein the T lymphocyte is an effector T lymphocyte.

10. The genetically engineered cell of any one of claims 1-7, wherein the T lymphocyte is a memory T lymphocyte.

11. The genetically engineered cell of any one of claims 1-7, wherein the T lymphocyte is a regulatory T lymphocyte (Treg).

12. The genetically engineered cell of claim 1, wherein the cell is a B-lymphocyte.

13. The genetically engineered cell of claim 1, wherein the cell is a natural killer (NK) cell.

14. The genetically engineered cell of any one of claims 1-13, wherein the hematopoietic stem cell mobilization comprises administering to the subject etoposide, plerixafor, cyclophosphamide, and/or granulocyte colony-stimulating factor (G-CSF).

15. The genetically engineered cell of any one of claims 1-14, wherein the chimeric antigen receptor is a first generation CAR.

16. The genetically engineered cell of any one of claims 1-14, wherein the chimeric antigen receptor is a second generation CAR.

17. The genetically engineered cell of any one of claims 1-14, wherein the chimeric antigen receptor is a third generation CAR.

18. The genetically engineered cell of any one of claims 1-17, wherein the lineage-specific cell-surface antigen is CD33, CD30, CD38, CD123, CLL-1, CD5, CD6, CD7, CD19, or BCMA.

19. The genetically engineered cell of any one of claims 1-18, wherein the hyperproliferative disease is a hematopoietic malignancy.

20. The genetically engineered cell of claim 19, wherein the hematopoietic malignancy is a myeloid malignancy.

21. The genetically engineered cell of claim 19 or 20, wherein the hematopoietic malignancy is Hodgkin's lymphoma, non-Hodgkin's lymphoma, leukemia, or multiple myeloma.

22. The genetically engineered cell of claim 21, wherein the leukemia is acute myeloid leukemia, acute lymphoid leukemia, myelodysplastic syndrome, chronic myelogenous leukemia, acute lymphoblastic leukemia or chronic lymphoblastic leukemia, or chronic lymphoid leukemia.

23. The genetically engineered cell of any one of claims 19-22, wherein the hematopoietic malignancy is acute myeloid leukemia.

24. The genetically engineered cell of any one of claims 19-22, wherein the hematopoietic malignancy is myelodysplastic syndrome.

25. The genetically engineered cell of claim 19, wherein the hematopoietic malignancy is a lymphoid malignancy.

26. The genetically engineered cell of any one of claims 1-25 for administration to a subject in need thereof, wherein the subject has, or has been diagnosed with a hematopoietic malignancy, and has received a hematopoietic stem cell transplant comprising genetically engineered stem cells that have reduced expression or lack expression of the lineage-specific cell surface antigen or express a variant form of the lineage-specific cell-surface antigen that is not recognized or is recognized at a reduced level by the CAR.

27. A method, comprising
contacting a mobilized lymphocyte obtained from a first subject with a heterologous nucleic acid encoding a chimeric antigen receptor (CAR) targeting a lineage-specific cell-surface antigen associated with a hyperproliferative disease, thereby producing a genetically engineered lymphocyte expressing the CAR.

28. The method of claim 27, further comprising administering the genetically engineered lymphocyte, or a descendant thereof, to a second subject,
wherein the second subject is in need thereof.

29. The method of claim 28, wherein the second subject has, or has been diagnosed with, the hyperproliferative disease.

30. The method of claim 28 or 29, wherein the first subject is different from the second subject.

31. The method of claim 28 or 29, wherein the first subject is the same as the second subject.

32. The method of any one of claims 28-31, wherein the subject in need of administering the genetically engineered lymphocyte, or a descendant thereof, has received a hematopoietic stem cell transplant comprising genetically engineered hematopoietic stem cells that have reduced expression or lack expression of the lineage-specific cell surface antigen, or express a variant form of the lineage-specific cell-surface antigen that is not recognized or is recognized at a reduced level by the CAR.

33. The method of any one of claims 28-31, further comprising
administering a hematopoietic stem cell transplant to the subject in need thereof after administration of the genetically engineered lymphocyte, or a descendant thereof;
wherein the transplant comprises genetically engineered hematopoietic stem cells that have reduced expression or lack expression of the lineage-specific cell surface antigen, or express a variant form of the lineage-specific cell-surface antigen that is not recognized or is recognized at a reduced level by the CAR.

34. The method of any one of claims 28-31, wherein the genetically engineered lymphocyte, or a descendant thereof, is administered in combination with a hematopoietic stem cell transplant comprising genetically engineered hematopoietic stem cells that have reduced expression or lack expression of the lineage-specific cell surface antigen, or express a variant form of the lineage-specific cell-surface antigen that is not recognized or is recognized at a reduced level by the CAR.

35. The method of any one of claims 27-34, further comprising
contacting a hematopoietic stem cell obtained from the first subject with an RNA-guided nuclease and guide RNA or a nucleic acid encoding the same, wherein the guide RNA targets a gene encoding the lineage-specific cell-surface antigen,
thereby producing a genetically engineered hematopoietic stem cell that has reduced expression or lacks expression of the lineage-specific cell-surface antigen or expresses a variant form of the lineage-specific cell-surface antigen,
wherein the genetically engineered hematopoietic stem cell is not targeted by the CAR.

36. The method of claim 35, further comprising administering the genetically engineered hematopoietic stem cell, or a descendant thereof, to a subject in need thereof.

37. The method of any one of claims 27-36, wherein the mobilized lymphocyte is a T lymphocyte.

38. The method of claim 37, wherein the T lymphocyte expresses CD3, CD4, and/or CD8.

39. The method of claim 37 or 38, wherein the T lymphocyte expresses CD4 and CD8.

40. The method of any one of claims 37-39, wherein the T lymphocyte expresses PD1.

41. The method of any one of claims 37-40, wherein the T lymphocyte is an alpha/beta T lymphocyte.

42. The method of any one of claims 37-40, wherein the T lymphocyte is a gamma/delta T lymphocyte.

43. The method of any one of claims 37-42, wherein the T lymphocyte is a naïve T lymphocyte.

44. The method of any one of claims 37-42, wherein the T lymphocyte is an effector T lymphocyte.

45. The method of any one of claims 37-42, wherein the T lymphocyte is a memory T lymphocyte.

46. The method of any one of claims 37-42, wherein the T lymphocyte is a regulatory T lymphocyte (Treg).

47. The method any one of claims 27-36, wherein the mobilized lymphocyte is a B-lymphocyte.

48. The method of any one of claims 27-36, wherein the mobilized lymphocyte is a natural killer (NK) cell.

49. The method of any one of claims 27-48, wherein the chimeric antigen receptor is a first generation CAR.

50. The method of any one of claims 27-48, wherein the chimeric antigen receptor is a second generation CAR.

51. The method of any one of claims 27-48, wherein the chimeric antigen receptor is a third generation CAR.

52. The method of any one of claims 27-51, wherein the lineage-specific cell-surface antigen is CD33, CD30, CD38, CD123, CLL-1, CD5, CD6, CD7, CD19, or BCMA.

53. The method of any one of claims 27-52, wherein the hyperproliferative disease is a hematopoietic malignancy.

54. The method of claim 53, wherein the hematopoietic malignancy is a myeloid malignancy.

55. The method of claim 53 or 54, wherein the hematopoietic malignancy is Hodgkin's lymphoma, non-Hodgkin's lymphoma, leukemia, or multiple myeloma.

56. The method of claim 55, wherein the leukemia is acute myeloid leukemia, myelodysplastic syndrome, acute lymphoid leukemia, chronic myelogenous leukemia, acute lymphoblastic leukemia or chronic lymphoblastic leukemia, or chronic lymphoid leukemia.

57. The genetically engineered cell of any one of claims 53-56, wherein the hematopoietic malignancy is acute myeloid leukemia.

58. The genetically engineered cell of any one of claims 53-56, wherein the hematopoietic malignancy is myelodysplastic syndrome.

59. The method of any one of claims 27-52, wherein the hematopoietic malignancy is a lymphoid malignancy.

60. The method of any one of claims 35-59, wherein the RNA-guided nuclease is a CRISPR/Cas nuclease.

61. The method of claim 60, wherein the CRISPR/Cas nuclease is a Cas9 nuclease.

62. The method of claim 60, wherein the CRISPR/Cas nuclease is an SpCas nuclease.

63. The method of claim 60, wherein the CRISPR/Cas nuclease is an SaCas nuclease.

64. The method of claim 60, wherein the CRISPR/Cas nuclease is a Cpf1nuclease.

65. The method of any one of claims 35-64, wherein the nucleic acid encoding the guide RNA and/or the RNA-guided nuclease is an RNA, preferably an mRNA or an mRNA analog.

66. The method of any one of claims 35-65, wherein the guide RNA comprises one or more nucleotide residues that are chemically modified.

67. The method of any one of claims 35-66, wherein the guide RNA comprises one or more nucleotide residues that comprise a 2′O-methyl moiety.

68. The method of any one of claims 35-67, wherein the guide RNA comprises one or more nucleotide residues that comprise a phosphorothioate.

69. The method of any of claims 35-68, wherein the guide RNA comprises one or more nucleotide residues that comprise a thioPACE moiety.

70. A cell population comprising a plurality of the genetically engineered cells of any of claims 1-26, or produced, obtained, or obtainable by the method of any of claims 27-69.

71. A method comprising administering the cell population of claim 70, to a subject in need thereof.
